SEQUENCE LISTING (34 sequences)

SEQ ID NO: 1 - DNA, 535 bp, Simian virus 40
tgatatctgcagaattccaccacactggactagtggatccgagctcggtaccaagcttacctagccagcttgggtctccctataggcgcgccagttcgcgatgctgatcacgtagatcttgcatcgataagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcagaaggtacctaaccaagttcctctttcagaggttatttcaggcc

SEQ ID NO: 2 - DNA, 43 bp, Artificial Sequence
  Primer containing restriction site
ccgctcgagttgcggccgctgtgccttctagttgccagccatc

SEQ ID NO: 3 - DNA, 34 bp, Artificial Sequence
  Primer containing restriction site
ggtaccatagagcccaccgcatccccagcatgcc

SEQ ID NO: 4 - DNA, 52 bp, Artificial Sequence
  Primer containing restriction site
ggccgctttattaattaagccctgcaggttgtttaaacttggcgcgccttat

SEQ ID NO: 5 - DNA, 50 bp, Artificial Sequence
  Primer containing restriction site
cgataaggcgcgccaagtttaaacaacctgcagggcttaattaataaagc

SEQ ID NO: 6 - DNA, 28 bp, Artificial Sequence
  Primer containing restriction site
cgggatccagacatgataagatacattg

SEQ ID NO: 7 - DNA, 54 bp, Artificial Sequence
  Fragment containing restriction sites
ggggaccactttgtacaagaaagctgggtttacacggcgatctttccgcccttc

SEQ ID NO: 8 - DNA, 33 bp, Artificial Sequence
  Primer containing restriction site
tggcgcgcctatagggagacccaagctggctag

SEQ ID NO: 9 - DNA, 38 bp, Artificial Sequence
  Primer containing restriction site
caatcataccgtttaaacgaaccgcgggccctctagac

SEQ ID NO: 10 - DNA, 59 bp, Artificial Sequence
  Primer containing restriction site
ggggacaagtttgtacaaaaaagcaggctatggaagacgccaaaaacataaagaaaggc

SEQ ID NO: 11 - DNA, 54 bp, Artificial Sequence; identical to SEQ ID NO: 7
  Primer containing restriction site

SEQ ID NO: 12 - DNA, 407 bp, Simian virus 40
aagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcagaaggtacctaaccaagttcctctttcagaggttatttcaggcc

SEQ ID NO: 13 - DNA, 42 bp, Artificial Sequence
  Probes and Primers
atagtttagcggccgcaacttgtttattgcagcttataatgg

SEQ ID NO: 14 - DNA, 40 bp, Artificial Sequence
  Probes and Primers
ttggcgcgccatggaagacgccaaaaacataaagaaaggc

SEQ ID NO: 15 - DNA, 36 bp, Artificial Sequence
  Probes and Primers
cccttaattaattacacggcgatctttccgcccttc

SEQ ID NO: 16 - DNA, 27 bp, Artificial Sequence
  Probes and Primers
gggatccagacatgataagatacattg

SEQ ID NO: 17 - DNA, 44 bp, Artificial Sequence
  Probes and Primers
atagtttagcggccgcaatgaatgcaattgttgttgttaacttg

SEQ ID NO: 18 - DNA, 51 bp, Artificial Sequence
  Probes and Primers
gccgctttattaattaagccctgcaggttgtttaaacttggcgcgccttat

SEQ ID NO: 19 - DNA, 50 bp, Artificial Sequence
  Probes and Primers
cgaaataattaattcgggacgtccaacaaatttgaaccgcgcggaatagc

SEQ ID NO: 20 - DNA, 14 bp, Artificial Sequence
  Probes and Primers
cggcggccgcgtac

SEQ ID NO: 21 - DNA, 43 bp, Artificial Sequence
  Probes and Primers
ttggcgcgcctcaatattggccattagccatattattcattgg

SEQ ID NO: 22 - DNA, 44 bp, Artificial Sequence
  Probes and Primers
gctaggtcggaggcgccggcccttgccacgtaaccttcgaacag

SEQ ID NO: 23 - DNA, 38 bp, Artificial Sequence
  Probes and Primers
ccacccaagcttatgaccgagtacaagcccacggtgcg

SEQ ID NO: 24 - DNA, 38 bp, Artificial Sequence
  Probes and Primers
cgtactgggcgttcgggccacggactgagctcgcctat

SEQ ID NO: 25 - DNA, 37 bp, Artificial Sequence
  Probes and Primers
caaccgctcgagctgtgccttctagttgccagccatc

SEQ ID NO: 26 - DNA, 31 bp, Artificial Sequence
  Probes and Primers
cccctacgccacccgagataccccatggggc

SEQ ID NO: 27 - DNA, 40 bp, Artificial Sequence
  Probes and Primers
accatggataaagttttaaacagagaggaatctttgcagc

SEQ ID NO: 28 - DNA, 33 bp, Artificial Sequence
  Probes and Primers
ttatgtttcaggttcagggggaggtgtgggagg

SEQ ID NO: 29 - DNA, 53 bp, Artificial Sequence
  Probes and Primers
tcctgcaggcggggtaccctagtctagactagccgcggggagtttaaacagct

SEQ ID NO: 30 - DNA, 53 bp, Artificial Sequence
  Probes and Primers
gtttaaactccccgcggctagtctagactagggtaccccgcctgcaggagtac

SEQ ID NO: 31 - DNA, 34 bp, Artificial Sequence
  Probes and Primers
gcaggctaccatggataaagttttaaacagagag

SEQ ID NO: 32 - DNA, 55 bp, Artificial Sequence
  Probes and Primers
gaaacctccgaagaccctacgttgactctaaggttggataccttgactacttacc

SEQ ID NO: 33 - DNA, 56 bp, Artificial Sequence
  Probes and Primers
ctttggaggcttctgggatgcaactgagattccaacctatggaactgatgaatggg

SEQ ID NO: 34 - DNA, 33 bp, Artificial Sequence
  Probes and Primers
aggaatgttgtacaccatgcattttaaaaagtc

(19) United States
(12) Patent Application Publication
(10) Pub. No.: US 2022/0105164 A1
(43) Pub. Date: Apr. 7, 2022

(54) Method for Restoring Immune Tolerance In Vivo

(71) Applicant: Amarna Holding B.V., Leiden (NL)

(72) Inventor: Petrus Theodorus De Haan, Oegstgeest (NL)

(21) Appl. No.: 17/497,354

(22) Filed: Oct. 8, 2021

Related U.S. Application Data
(63) Continuation-in-part of application No. 15/038,443, filed on May 20, 2016, now abandoned.

(30) Foreign Application Priority Data
Nov. 22, 2013 (EP) 13194126.2
Jan. 29, 2014 (EP) 14153144.2

(51) Int. Cl. A61K 39/00; C07K 14/005; A61P 37/02

(52) U.S. Cl. CPC A61K 39/0008; A61K 2039/5254; A61P 37/02; C07K 14/005

(57) ABSTRACT

The present invention provides a method for restoring immune tolerance in vivo. The invention relates to the use of a recombinant gene encoding auto-antigens or parts thereof for restoring immune tolerance to the auto-antigens in vivo, under transcriptional control of polyomaviral early and late promoters. In a preferred embodiment, the invention relates to the use of recombinant polyomaviral gene delivery vector particles, such as simian virus 40 (SV40) viral vector particles encoding one or multiple auto-antigens or parts thereof under transcriptional control of the SV40 early and late promoter, for restoring immune tolerance to the auto-antigens in vivo. The invention also relates to compositions comprising recombinant genes or polyomaviral vectors and uses thereof as treatment for degenerative or dystrophic diseases.

### CROSS-REFERENCE TO RELATED APPLICATIONS

This application is a continuation-in-part U.S. application Ser. No. 15/038,433, Filed on May 20, 2016, published as US 2016/0287685 on Oct. 6, 2016, which is a national phase entry of International Patent Application PCT/EP2014/075346, filed Nov. 22, 2014, designating the United States of America and published in English as International Patent Publication WO 2015/075213 Al on May 28, 2015, which claims the benefit under Article 8 of the Patent Cooperation Treaty to European Patent Application Serial No. 14153144.2, filed Jan. 29, 2014, and to European Patent Application Serial No. 13194126.2, filed Nov. 22, 2013.

### TECHNICAL FIELD

The present invention relates to improved methods for the production of viral particles, viral vectors, viral vector particles and recombinant proteins. In particular, the invention relates to improved methods for the production of recombinant polyomaviral vector particles and polyomaviral vector producer cell lines. More in particular, the invention relates to methods for the production of simian polyomaviral vectors such as simian virus 40 (SV40) viral vectors. The invention also relates to compositions comprising viral vectors and uses thereof and viral vector particles to treat genetic disorders, transplant rejection, auto-immune diseases, infectious diseases, allergies or cancer.

The present invention relates to an improved method for the restoration of immune tolerance in vivo. In particular, the invention relates to the use of a recombinant gene encoding one or multiple auto-antigens or antigenic parts thereof to restore immune tolerance to the antigens in vivo. The invention also relates to compositions comprising a recombinant gene encoding one or multiple auto-antigens or antigenic parts thereof and uses thereof as a treatment for autoimmune diseases.

### BACKGROUND

The immune system consists of an intricate network of tissues, cells, and molecules responsible for responding to cell death, wounding or pathogen infection and maintaining the body's homeostasis. During homeostasis, dying cells that undergo programmed cell death (apoptosis) are removed by specific cells of the immune system, e.g., immature macrophages and dendritic cells, and are replenished by cells descending from tissue-resident stem cells. The cellular components are processed by the immature macrophages and dendritic cells into auto-antigens that are presented on major histocompatibility complex (MHC) molecules to cells of the adaptive immune system (signal 1). Macrophages and dendritic cells therefore represent the main antigen-presenting cells in the body. Antigen presentation takes place in the presence of tolerogenic co-stimulatory molecules (signal 2) such as B7-1 (CD80), B7-H1 (CD274), B7-DC, B7-H3 and B7-H4 and tolerogenic chemokines as well as cytokines (signal 3) such as interleukin 10 (IL10) and transforming growth factor beta. Immature antigen-presenting cells carry chemokine receptors CCR1, CCR2, CCR5, CCR6 and CXCR1; antigen receptors of the C-type lectin family such as DEC-205, the dendritic cell immunoreceptor, dectin-2 and C-type lectin receptor 1; the macrophage mannose receptor and Fc receptors for immunoglobulins such as the Fc gamma receptor and Fc epsilon receptor; toll-like receptors; integrins and heat shock protein receptors. In addition, immature antigen-presenting cells are characterized by having high levels of intracellular MHC, whereas they have low levels of B7-2 (CD86), CD40, CD25 and CD83 on their surface. As a result of these homeostatic cell-cell interactions the immune system is in the immune tolerance state.

Following wounding or pathogen infection, injured or infected cells undergo necrosis and release danger-associated or pathogen-associated molecular patterns, respectively. These molecular patterns induce local tissue inflammation at the site of injury or infection. Necrotic cells are removed by antigen-presenting cells that subsequently mature and they present the processed (self) antigens to cells of the adaptive immune system in the presence of proinflammatory signals 2 and 3. During their conversion from immature to mature cells, antigen-presenting cells undergo a number of phenotypical and functional changes.

Antigen-presenting cell maturation involves: (i) redistribution of MHC molecules from intracellular endocytic compartments to the cell surface; (ii) down-regulation of antigen internalization; (iii) increase in the surface expression of co-stimulatory molecules such as B7-2, CD40 and B7-H2 (CD275) (signals 2); (iv) morphological changes (e.g., formation of dendrites); (v) cytoskeleton re-organization; (vi) secretion of chemokines such as CCL17, CCL18, CCL22 and cytokines such as IL3, IL4, IL5, IL6, IL12, IL13, IL14, interferon alpha, interferon beta, interferon gamma and tumor necrosis factor alpha (signals 3) and (vii) surface expression of adhesion molecules and chemokine receptors such as CXCR4 and CCR7.

Mature antigen-presenting cells are characterized by having low levels of Fc receptors, intercellular adhesion molecule-1 and CD58 on their surface. Cells of the adaptive immune system, mainly consisting of T- and B-cells activated by interacting with the mature antigen-presenting cells, now temporarily break the immune tolerance and destruct/clear the injured or infected cells in an antigen-specific manner. After a normal wound repair response, thus in the absence of the danger-associated or pathogen-associated molecular patterns, the immune tolerance is restored, again in an antigen-specific manner.

In patients with degenerative diseases, such as dystrophic diseases and chronic viral infections, at a certain point in time specific tissues/organs (brain, muscle, blood, liver, eye, skin etc.) start to accumulate necrotic cells. The necrotic cells induce non-resolving inflammation, wherein chronic activation of cells of the adaptive immune system leads to a continuous and local breaking of the immune tolerance and to a destruction of cells of the inflamed tissue in an antigen-specific manner. The activated cytotoxic T lymphocytes destruct their target cells by inducing cell necrosis. The necrotic cells on their turn further enhance tissue inflammation. In addition, chronic inflammation results in amyloid plaque formation and tissue scarification due to collagen deposition. It has been generally accepted that this auto-antigen-specific chronic stimulation of the adaptive immune response is the major driver of the tissue/organ destruction (C. Nathan and A. Ding, Cell 140:871-882, 2010).

Examples of genetic disorders resulting in a degenerative disease are neurological diseases such as Huntington disease and the familial forms of Alzheimer's dementia and Parkinson disease; the familial muscular dystrophies such as Duchenne muscular dystrophy, Becker muscular dystrophy, Miyoshi myopathy, Limb-girdle muscular dystrophy, Congenital muscular dystrophy, Distal muscular dystrophy, Emery-Dreyfuss muscular dystrophy, Fascio-scapulohumeral muscular dystrophy, Myotonic muscular dystrophy and Oculopharyngeal muscular dystrophy; ophthalmological dystrophies such as Retinitis pigmentosa, Leber's congenital amaurosis, Stargardt macular dystrophy, Achromatopsia, Retinoschisis and Vitelliform macular dystrophy.

In sporadic cases, these diseases are acquired diseases. This means that in genetically predisposed individuals at a certain point in time, unknown environmental stimuli, mimicking danger- or pathogen-associated molecular patterns, initiate the process of nonresolving inflammation of specific tissues. In these cases the diseases are named autoinflammatory or autoimmune diseases.

With our increased knowledge in human immunology the list of autoimmune diseases is expanding rapidly and currently includes, but is not limited to, neurological diseases such as Alzheimer's dementia, amyotrophic lateral sclerosis, autism, bipolar disorder, depression, epilepsy, narcolepsy, Lyme disease, multiple system atrophy, multiple sclerosis, myasthenia gravis, neuromyelitis optica, Parkinson's disease and schizophrenia; metabolic diseases such as type 1 diabetes, type 2 diabetes, Hashimoto' s thyroiditis and obesity; muscular diseases such as polymyositis, dermatomyositis and inclusion body myositis; ophthalmological diseases such as autoimmune retinopathy, age-related macular degeneration, glaucoma and uveitis; gastro-enteric diseases such as celiac disease and inflammatory bowel diseases including Crohn's disease and ulcerative colitis; dermatological diseases such as psoriasis, scleroderma, Sjogren's syndrome and Vitiligo; cardio-vascular diseases such as atherosclerosis, cardiomyopathy and Coxsackie myocarditis; orthopedic diseases such as rheumatoid arthritis, rheumatic fever and lupus erythematosus; and pulmonary diseases such as chronic obstructive pulmonary disease and asthma. Furthermore, amyloidosis, autoimmune hepatitis, Chagas disease, Endometriosis, Goodpasture's syndrome, Hashimoto's encephalitis, idiopathic thrombocytopenic purpura, Kawasaki syndrome, Meniere disease, neutropenia, 4estless legs syndrome and thrombocytopenic purpura are autoimmune diseases.

Current treatments for patients with a degenerative or dystrophic disease non-specifically suppress inflammation and immunity, and thus only alleviate the symptoms and delay the progression to disabling stages. Furthermore, long term use of immuno-suppressing medication coincides with often severe adverse side effects and enhances the risk of developing autoimmune processes in other tissues.

There is, therefore, an urgent need for alternative treatments that specifically inhibit tissue destruction by activated cells of the adaptive immune system, leaving the general immune response unaffected. In principle, this should be feasible since autoimmune diseases are acquired diseases. Restoring immune tolerance to the major auto-antigens involved in the autoimmune tissue destruction has been a longstanding goal in autoimmunity research. This has been attempted by administration of the auto-antigens or peptide fragments derived thereof to patients. However, due to the fact that these proteins or peptides are not efficiently delivered to the proper antigen-presenting cells, that they lack the proper accompanying signals for reverting the immune response and/or that they are rapidly degraded in the body, such approaches have been adopted with limited success.

An efficient way to instruct cells of the immune system to suppress an autoimmune response is to let them produce the auto-antigens involved in the autoimmune disease by introducing the antigen-encoding genes into these cells. Transgenic mice expressing myelin basic protein (MBP) in the liver are protected from MBP-induced experimental autoimmune encephalitis (EAE), a neurological disease in mouse with symptoms that resemble multiple sclerosis in humans. Transgenic mice expressing MBP in the skin and thymus however, are not protected from developing EAE, indicating that the liver is an important tolerogenic organ (S. Lueth et al., The Journal of Clinical Investigation 118:3403-3410, 2008). In a therapeutic setting, the auto-antigen-encoding therapeutic genes can be administered as naked molecules or as nucleic acids packaged in lipid and/or proteinaceous compounds. In mice with myelin oligodendrocyte glycoprotein (MOG)-induced EAE model, injection of an expression plasmid encoding MOG or MBP reduced the clinical and histopathological signs of EAE in both prophylactic and therapeutic settings (H. Garren, Expert Opinion Biological Therapy 8:1539-1550, 2008; N. Fissolo et al., Journal of Neuroinflammation 9:139-152, 2012; U.S. 2010/0160415). In non-obese diabetes (NOD) mice, the most commonly used animal model for type 1 diabetes in humans, injection of expression plasmids encoding preproinsulin or glutamic acid decarboxylase 65 (GAD65), the major auto-antigens involved in autoimmune destruction of the pancreatic insulin-producing cells, results in both prevention and suppression of autoimmune diabetes (M. C. Johnson et al., Human Vaccines 7:27-36, 2011; Y. Guan et al., Diabetes Research Clinical Practice 95:93-97, 2012). This DNA vaccination approach however, only works in mice. In all other mammalian species including humans, cells do not take up and express extracellular nucleic acids.

Over the last decade, much effort has been dedicated to the development of efficient gene or nucleic acid delivery technologies for introduction and proper expression of genes or nucleic acids in target cells. Therapeutic genes or nucleic acids can be used to restore malfunctioning genes to treat genetic disorders, to induce an immune response to treat cancer and infectious diseases or to suppress an immune response e.g. for inducing/restoring immune tolerance to prevent transplant rejection or to treat autoimmune diseases and allergies. The therapeutic genes or nucleic acids can be administered as naked molecules or as nucleic acids packaged in lipid and/or proteinaceous compounds.

Since viruses evolved to deliver and express their genetic information into host target cells, viral vectors are currently the most effective gene delivery vehicles. Besides gene therapy applications viral vectors have been shown to be highly effective in modulating immune responses in animal models of autoimmune diseases.

Since viruses evolved to deliver and express their genetic information into their host target cells, viral vectors have been explored as gene delivery vehicles and were found to be by far the most effective means of delivering genetic information into a living cell. A number of viral vector gene delivery systems have been developed and tested in preclinical and clinical trials. These trials revealed that the currently used vectors, which are derived from adenoviruses, poxviruses, herpesviruses, alphaviruses, retroviruses, parvoviruses and polyomaviruses, are generally safe to use and efficient in delivering therapeutic genes to target cells.

In mice with rheumatoid arthritis induced by subcutaneous administration of type II collagen, the disease progression was halted by intravenous administration of a lentiviral vector encoding type II collagen (T. Eneljung et al., Clinical and Developmental Immunology 2013:11, 2013).

In NOD mice that have been administered with adeno-associated virus (AAV) or lentiviral vectors encoding preproinsulin or GAD65 immune tolerance to the pancreatic insulin-producing cells was efficiently restored and the development of type 1 diabetes was prevented (M. C. Johnson et al., Human Vaccines 7:1-10, 2011; R. M. Jindal et al., International Journal of Experimental Diabetes Research 2:129-138, 2001; C. Dresch et al., Journal of Immunology 181:4495-4506, 2008; G. Han et al., Journal of Immunology 174:4516-4524, 2005; G. Han et al., Immunology Letters 115:110-116, 2008; B. Xu and D. W. Scott, Clinical Immunology 111:47-52, 2004). Implantation of bone marrow-derived stem cells or hepatic cells transduced ex vivo with lentiviral vectors encoding the major MOG epitopes in mice, protected the treated animals from MOG-induced EAE (B. E. Hoffman and R. W. Herzog, The Journal of Clinical Investigation 118:3271-3273, 2008; D. De Andrade Pereira et al., Gene Therapy 20:556-566, 2013). Intrathymical delivery of MOG using a lentiviral vector induced central immune tolerance to MOG, which prevented mice from developing EAE upon challenge with MOG (G. Marodon et al., Blood 108:2972-2978, 2006). The progression of EAE in mice that received MOG first could, however, not be stopped using this central tolerization approach (C. Siatskas et al., Molecular Therapy 20:1349-1359, 2012). This study demonstrates that auto-antigens need to be expressed and exposed to antigen presenting cells in the periphery preferably in tolerogenic organs, in order to revert the pre-existing immune response into an immune tolerance response.

These and other animal studies have demonstrated that an active and specific immune tolerance response can be induced against a self-protein when the gene encoding the self-protein is expressed ectopically under transcriptional control of liver-specific promoters derived from host genes, such as the albumin, the DC190, the alpha anti-trypsin (AAT), the C-reactive protein (CRP) or the dendritic cell-specific transmembrane protein (DC-STAMP) promoters (A. Follenzi et al., Blood 103:3700-3709, 2004; C. Siatskas et al., Molecular Therapy 20:1349-1359, 2012; S. Lueth et al., The Journal of Clinical Investigation 118:3403-3410, 2008; D. De Andrade Pereira et al., Gene Therapy 20:556-566, 2013; B.E. Hoffman and R. W. Herzog, The Journal of Clinical Investigation 118:3271-3273, 2008). In addition, liver-specific expression of coagulation factors VIII or IX introduced by AAV or lentiviral vectors efficiently restored immune tolerance to the coagulation factors in dogs and mice with pre-existing immunity to these factors (C. Siatskas et al., Molecular Therapy 20:1349-1359, 2012; J. D. Finn et al., Blood 116:5842-5848, 2010). However, recent clinical gene therapy studies conducted demonstrated that the organ-specific and host-derived promoters used to control transcription of the therapeutic genes are not potent enough to yield therapeutic levels of self-proteins in the treated patients (A. C. Nathwani et al., New England Journal of Medicine 365:2357-2365, 2011).

Moreover, ectopic expression of genes encoding self-proteins under transcriptional control of strong constitutive viral promoters such as the cytomegalovirus (CMV) immediate early promoter or retroviral long terminal repeat promoters leads to the induction of an adaptive immune response directed against the self-protein (A. Follenzi et al., Blood 103:3700-3709, 2004; H. J. Ko et al., Autoimmunity 44:177-187, 2011). For example, it has been found that macaques develop an autoimmune anemia upon gene therapy with adeno-associated virus vectors encoding erythropoietin (G. Gao et al., Blood 103:3300-3302, 2004; P. Chenuaud et al., Blood 103:3303-3304, 2004). Erythropoietin was over-expressed in macaques under transcriptional control of the cytomegalovirus immediate early promoter or a doxycyclin-inducible promoter derived from the HIV-1 long terminal repeat. These animals were found to develop an adaptive immune response against endogenous erythropoietin or cells producing erythropoietin. So immune therapy wherein auto-antigens are over-expressed may induce rather than cure an autoimmune disease.

Although viral vector-based tolerization approaches to treat degenerative diseases using organ-specific and host-derived promoters driving expression of auto-antigens look promising, it has become obvious from the above described prior art that there is an unmet need for treatments that restore immune tolerance to the auto-antigens.

A major disadvantage of the currently used viral gene delivery vectors is the fact that they cannot be produced in sufficient amounts to treat significant numbers of patients. The majority of viral vectors is produced by transfecting producer cells with plasmid DNA encoding the vector and the vector components. This generally yields 1 to 10 million vector particles per milliliter cell culture volume. In clinical trials, generally 1×10¹⁰ to 1×10¹² vector particles have to be administered to a patient in order to accomplish beneficial clinical effects. This means that in order to treat 1000 patients, more than 1 million liters of cell culture are required to yield sufficient amounts of vector particles.

In addition, preclinical and clinical trials revealed that most of the tested viral gene delivery vectors such as adenoviral, poxviral, herpesviral, alphaviral and retroviral vectors induce a strong immune response in patients, directed to viral vector components and the therapeutic gene products. As a consequence, these vectors can only be administered a single time to a patient, whereas the expression levels of the introduced therapeutic gene rapidly decline. Viral vectors derived from adeno-associated virus (AAV) do not induce immune responses in animals and are immunologically inert. However, the majority of the human population encountered wildtype AAV together with its helper virus, e.g. adenovirus and as a result developed a strong CTL memory against the AAV capsid proteins. As a consequence, AAV-transduced cells are rapidly removed and the expression levels of the therapeutic gene or nucleic acid introduced by an AAV viral vector rapidly decline.

The yields of recombinant proteins produced in mammalian cells compared to those produced in prokaryote cells are in general low, despite the use of strong promoters and/or multicopy transgene insertions or other ways to enhance the transcription. Viral replication competent vectors or replicons have been used for a long time as expression systems for the production of recombinant proteins in mammalian cells. The target gene in such vectors can be expressed under transcriptional control of viral promoters whereby the desired mRNAs may accumulate to extremely high levels in the cytoplasm early after transfection, yielding large amounts of target protein. So far the successes with replicon-based expression systems have been limited. Replicon systems based on RNA viruses in general produce recombinant proteins for only a short period of time, whereas those derived from DNA viruses in general do not replicate well in the commercially used cell lines.

To our knowledge, there is only one viral gene delivery vector that is immunologically inert in humans and that can be produced in sufficient amounts to treat a significant number of patients. Moreover, this viral gene delivery vector can be employed as a replicon system for the production of recombinant proteins in mammalian cell lines. This viral vector system is derived from simian virus 40 (SV40), a simian polyomavirus.

Polyomaviruses are comprised of a family of non-enveloped DNA viruses with icosahedral capsids. They are isolated from a variety of animal species including humans, monkeys, rodents and birds. Five human polyomaviruses have been described, termed BK, JC, WU, KI and Merkel Cell polyomavirus. Many monkey polyomaviruses have been described of which SV40 is the most well-known. SV40 replicates poorly in human cells and infections in humans are rare. Occasional SV40 infections occurred through transmission of the virus from monkeys to people living in close contact with these animals or through vaccination with batches of inactivated poliovirus particles contaminated with SV40.

SV40 has a 5.25 kilo base pairs long circular double stranded DNA genome. The SV40 genome consists of two regulatory regions, the promoter/origin region and the polyadenylation region. The promoter/origin region is 500 base pairs long and comprises two oppositely-directed promoters, the early and late promoter (SVEP and SVLP respectively), the origin of replication and the packaging signal. The polyadenylation region is 100 base pairs long and contains the polyadenylation signals of both the early and the late transcripts. SVEP drives expression of the early primary transcript that is spliced by host-encoded splicing factors into 2 different mRNAs encoding small and large tumor (T) antigens.

The large T antigen is the replicase-associated protein required for DNA replication and for activation of the SVLP. Although the precise role of the small T antigen in virus replication has remained unclear, small T antigen is required for the transformation of several mammalian cell types, in conjunction with large T antigen. The primary effects of small T antigen occur through its interaction with serine-threonine protein phosphatase 2A. The phosphatase 2A-binding domain of small T antigen is located at the unique carboxy-terminal end of the small T antigen.

It is well documented in the prior art that both large T antigen and small T antigen are required for efficient polyomavirus replication (Fahrbach K. M. et al., Virology 370 (2): 255-263, 2008).

SVLP drives expression of the late primary transcript that is spliced by host-encoded splicing factors into different mRNAs encoding the viral capsid proteins VP1, 2 and 3. The T antigens are the major and the capsid proteins the minor immunogenic components of polyoma viruses, eliciting cellular and humoral immune responses against SV40-infected cells.

The SV40 T antigens cooperatively immortalize primary mammalian cells, transform established mammalian cell lines and induce tumours in immuno-compromized young-borne rodents. A number of reports suggest that SV40 infections are associated with human malignancies, caused by the oncogenic activity of the chronically expressed T antigens (Butel J. S. and Lednicky J. A. Journal of the National Cancer Institute 91: 119-134, 1999).

Since expression of the viral capsid proteins is dependent on the presence of the large T antigen, T antigen-specific sequences have been deleted in polyoma viral vectors, not only for rendering the vectors replication-incompetent, but also to completely eliminate their immunogenicity in humans.

T antigen-deleted polyoma viral vectors derived from SV40 have been made and tested, in which the therapeutic genes or nucleic acids are expressed in trans in target cells under transcriptional control of the viral SVEP. Said vectors are known for a long time as potential vectors for gene transfer into a plurality of human tissues and cell types, for example, bone marrow (Rund D. et al, Human Gene Therapy 9: 649-657, 1998), the liver (Strayer D. S. and Zern M. A., Seminars in Liver Disease 19: 71-81, 1999) and dendritic cells (Vera M. et al., Molecular Therapy 12: 950-959, 2005).

Polyomaviral vectors, such as SV40, are known to infect non-dividing as well as actively dividing cells. Since the vectors lack the region encoding the T antigens and as a consequence do not express the viral capsid proteins, they are non-immunogenic (Strayer D. S. and Zern M. A., Seminars in Liver Disease 19: 71-81, 1999) allowing repeated administration to the same individual. Moreover, since the inserted therapeutic gene constructs are expressed under transcriptional control of SVEP, a weak but constitutive promoter, said vectors induce long-term expression of the therapeutic proteins in vivo. Thus, it is known that polyomaviral vectors, such as SV40-derived vectors are promising candidates for therapeutic gene or nucleic acid transfer that can be used for the above-mentioned applications.

Because of their replication potential, polyomavirus-based replicons are also of great interest to enhance the production of recombinant proteins such as antibodies, growth factors and hormones in mammalian cells.

T antigen-deleted SV40 particles have been produced in simian cells that are permissive for lytic growth of SV40 and that supply the T antigens in trans. SV40 vector packaging cell lines that are currently used are COS cell lines in particular COS-1 and COS-7 (Gluzman Y., Cell 23: 175-182, 1981). COS cell lines were generated by transformation of monkey CV1 cells with SV40 DNA. Another cell line that expresses the SV40 T antigen in trans is CMT4. The CV1-derived CMT cell lines were generated using SV40 DNA in which the T antigens were expressed under transcriptional control of the mouse metallothionein promoter (Gerard R. D. and Gluzman Y., Molecular and Cellular Biology 5: 3231-3240, 1985).

There is an important disadvantage however to the use of such cell lines. Passaging of T antigen-deleted SV40 vectors in the constructed packaging cell lines (COS or CMT) in many cases results in the appearance of wildtype replication-competent SV40 particles (Gluzman Y., Cell 23: 175-182, 1981; Oppenheim A. and Peleg A., Gene 77: 79-86, 1989; Vera M. et al., Molecular Therapy 10: 780-791, 2004).

This most likely occurs by nucleotide sequence homology-dependent recombination between the chromosomally inserted SV40-specific sequences and nuclear SV40 vector-specific sequences. The emergence of the replication competent wildtype virus particles and the presence of the T antigen oncoproteins in such conventional packaging cell lines have made the use of SV40 vectors for medical purposes impractical.

The human embryo kidney 293 (HEK293) cell line is semi-permissive to SV40 infection, which means that only a small percentage of infected cells support virus replication. The majority of cells are persistently infected and show very low levels of virus replication.

A derivative of the HEK293 cell line is the HEK293T cell line, expressing the SV40 early region under transcriptional control of the Rous sarcoma virus long terminal repeat promoter. It has been described that HEK293T cells express very low amounts of large T antigen and large amounts of small T antigen, due to a splicing bias in favour of the SV40 small T antigen mRNA. Vera et al. found that HEK293T poorly supports SV40 viral vector production (Vera M., et al., Molecular Therapy 10: 780-791, 2004).

Since the T antigen oncoproteins are present in HEK293T cells and there is a risk that replication competent SV40 viruses emerge, the use of this cell line for the production of SV40 vectors for medical purposes is undesired and impractical.

The HEK293TT cell line has been developed as a derivative of HEK293T, generated by stable transfection with a gene construct encoding the SV40 large T antigen. HEK293TT cells are used for the production of recombinant human papilloma virus (HPV) pseudo-vector particles. The recombinant HPV pseudo-vector particles are produced in HEK293TT by transfecting the cells with a plasmid that harbours the SV40 origin of replication and the HPV capsid genes and one that harbours the SV40 origin of replication and a HPV pseudo-genome (Buck C. B. et al., Methods in Molecular Medicine 119: 445-462, 2005).

Since HEK293TT as a derivative of HEK293T accumulates the small and large T antigen oncoproteins and poorly supports SV40 replication, the use of this cell line to produce recombinant SV40 vectors for medical purposes is also undesired and impractical.

WO 03/025189 describes packaging complementation cell lines that allow for the production of SV40 vector particles that are allegedly safe for medical use. However, the packaging cell lines described herein still accumulate significant amounts of the small and large T antigen oncoproteins.

Vera M. et al., Molecular Therapy 10: 780-791, 2004 showed that the production capacity of recombinant SV40 vector particles of interest in certain cell lines such as CMT4 and HEK293T can be very low and state that the cell lines described in WO 03/025189, such as COT-2 are also not effective as producer cell lines for the recombinant SV40 virus particles, possibly due to the splicing bias in favour of the small T antigen mRNA in these cell types.

WO 08/000779 describes a method to overcome the problem with the production of high titre stocks of suitable SV40 viral vectors using viral suppressors of RNA interference (RNAi), such as the vaccinia virus E3L and influenza A virus NS1 proteins. The packaging cell lines described in WO 08/000779 do not provide a solution to the disadvantages of the packaging cell lines of the prior art described herein above.

Chinese hamster ovary (CHO) cells have been provided with the mouse polyomavirus early region, resulting in CHOP cell lines (Heffernan and Dennis, Nucleic Acids Research 19: 85-92, 1991). A number of CHOP cell lines supported replication of plasmid CDM8 (invitrogen), a mammalian expression vector carrying the mouse polyomavirus origin of replication. The level of replication in the CHOP cell lines was not sufficient to make this system attractive for commercial application, possibly due to a splicing bias in favour of the small T antigen or middle T antigen mRNA in CHO cells.

There remains a desire in the art for efficient production systems for recombinant polyomavirus particles that are safe to use and yield high titers of viral vector particles. It is therefore an object of the present invention to provide methods for the safe and efficient production of polyomavirus particles and compositions obtainable therewith. It is appreciated that the methods of the present invention can also be used for the production of large amounts of recombinant proteins in mammalian cells.

### BRIEF SUMMARY

The above objects have been met by the present invention in that a method is provided for restoring immune tolerance in vivo. The invention relates to the use of a recombinant gene comprising a heterologous DNA sequence harboring the coding domain of one or multiple auto-antigens or antigenic parts thereof and wherein the heterologous DNA is expressed in a target cell under transcriptional control of a full-length polyomaviral early and late promoter, also often referred to as the polyomaviral intergenic region. This leads to restoring immune tolerance to the auto-antigens in vivo. In a preferred embodiment, the polyomaviral intergenic region is the SV40 intergenic region or full-length SV40 early and late promoter as disclosed in SEQ ID NO: 19. In another preferred embodiment, a promoter according to SEQ ID NO: 2 is used.

The above objects have been met by the present invention in that a method is provided for producing recombinant polyomaviral vector particles incapable of expressing a functional small T antigen comprising the steps of a) providing a cell line permissive to the wildtype polyomavirus, said cell line being capable of expressing a functional large T antigen but incapable of expressing a functional polyomaviral small T antigen, b) introducing into said cell line a polyomavirus DNA incapable of encoding a functional small T antigen, c) culturing said cells in a growth medium under conditions allowing the formation of recombinant polyomaviral vector particles and d) harvesting the recombinant polyomaviral vector particles from the cell culture.

The present invention therefore provides a method for restoring immune tolerance in vivo. The invention relates to the use of a recombinant gene encoding auto-antigens or antigenic parts thereof for restoring immune tolerance to the auto-antigens in vivo, under transcriptional control of polyomaviral early and/or late promoters. In a preferred embodiment, the invention relates to the use of recombinant polyomaviral gene delivery vector particles, such as simian virus 40 (SV40) viral vector particles encoding one or multiple auto-antigens or antigenic parts thereof under transcriptional control of polyomaviral early and late promoter such as the full-length SV40 early and late promoter, for restoring immune tolerance to the auto-antigens in vivo. The invention also relates to compositions comprising recombinant genes or polyomaviral vectors and uses thereof as treatment for degenerative or dystrophic diseases.

The recombinant polyomaviral vector particles produced by this method are incapable of expressing functional small T antigen and cannot revert into wildtype polyomavirus particles. This may be due to a complete lack of genes encoding a functional small T antigen, in the polyomaviral vector as well as in the polyomaviral vector producer cell line.

This now enables for the first time the preparation of a composition comprising a significant number of recombinant polyomaviral vector vector particles without a single wildtype polyomavirus particle. The viral vector particles produced in this method are unable to replicate in cells which are permissive for the wildtype polyomavirus and which do not express a functional large T antigen. Hence this composition is safe to use in medical treatments.

To date, it has not been possible to produce large quantities of polyomaviral vector particles, free of wildtype polyomavirus revertants or recombinants. With the use of the invention, it is now possible to obtain large quantities of uniform viral vector particles without a single wildtype virus being present. Accordingly, the invention relates to a composition comprising more than 10⁶ polyomavirus particles incapable of expressing a functional small T antigen and therefore incapable of replicating in large T antigen-deficient cells which are permissive for the wild type polyomavirus.

Cell lines for use in this invention may be conventional mammalian cell lines permissive for a polyomavirus that are genetically modified so that they express a functional polyomaviral large T antigen and do not express a functional polyomaviral small T antigen. Cell lines according to the invention may advantageously be used for the production of recombinant proteins since they are able to replicate circular DNA molecules harbouring the polyomavirus origin of replication.

The invention also relates to a composition as described above for use as a medicament.

In a preferred embodiment, a full-length polyomaviral early and late promoter is used, preferably the SV40 early and late promoter, (SEQ ID NO: 19 or SEQ ID NO:21).

### BRIEF DESCRIPTION OF THE DRAWINGS

FIGS. 1A-1C: Graphs showing the relation between suppression of RNA interference (0, 10, 50 and 100 nanograms of VP35) and expression of luciferase under the control of three different promoters: FIG. 1A, CMV immediate early promoter (pRL-CMV); FIG. 1B, SV40 early promoter (pGL3); and FIG. 1C, the full-length SV40 early and late promoter (SV Luc, SEQ ID NO: 19).

### DETAILED DESCRIPTION

The present inventor surprisingly found that human peripheral blood mononuclear cells (PBMCs) secreted tolerogenic cytokines upon transfection with an expression plasmid encoding an antigen under the control of a full-length polyomaviral early and late promoter.

The expression plasmids were constructed wherein the expression of firefly luciferase and myelin oligodendrocyte glycoprotein (MOG) was under the transcriptional control of the polyomaviral intergenic region. It was found that immune tolerance was induced against these antigens. This result shows that we were able to induce tolerance against auto-antigen as well as non-self antigen by expressing these antigens under the control of the polyomaviral intergenic region, comprising the full-length polyomaviral early and late promoter.

This is in contrast to a prejudice that viral promoters induce an adaptive immune response to a transgene product expressed under their transcriptional control. Without wanting to be bound by theory, we hypothesized that the main reason why viral promoters in general induce an immune response in vivo is that these viral promoters induce RNA interference, in which the messenger RNA molecules encoding the transgene products are degraded, resulting in the accumulation of short double-stranded RNA molecules in the cytoplasm. Double-stranded RNA molecules are highly effective inductors of inflammatory responses.

The present inventor has found that the large T antigen of a polyomavirus on its own promotes expression of the polyomaviral capsid proteins and that the polyomaviral small T antigen is not required for that purpose. This means that in the absence of polyomaviral T antigens in cells, the SVEP is a constitutive but weak promoter, compared to other viral promoters such as the cytomegalovirus (CMV) immediate early promoter, whereas the SVLP is shut-off at the transcriptional or post-transcriptional level. Surprisingly, in SV40-permissive cells, the SV40 large T antigen on its own was found to be capable of sustaining the multiplication of SV40 viral vector DNA and of activating SVLP, leading to the accumulation of capsid proteins and resulting in the efficient production of SV40 viral vector particles.

In the prior art, SV40 strains have been generated that are deficient in encoding the small T antigen. Gauchat et al. (Nucleic Acids Research 14: 9339-9351, 1988) describe an SV40 deletion mutant d1883 that lacks the small T antigen but produces a functional large T antigen in infected cells. When this mutant virus strain was used to infect monkey kidney cells and CV-1 cell cultures, the mutant virus strain was less efficient in inducing large T antigen-mediated cell division and subsequent virus replication than wildtype SV40. The authors concluded that the small T antigen has a helper function, assisting the large T antigen in inducing cell division and virus replication. This prior art thus teaches away from the present invention, since it shows that compared to cells infected with wildtype SV40, many cells infected with d1883 do not divide and do not produce virus particles. The absence of small T antigen in a cell is taught to be detrimental to viral vector production.

The Gauchat et al. publication is inconclusive on whether virus particles are produced or not. They merely measure the production of virus DNA in cells, which is not equivalent to the production of intact virus particles.

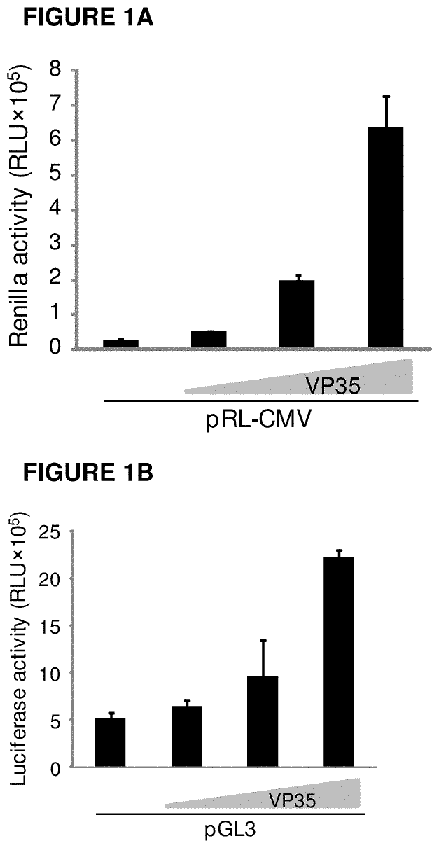

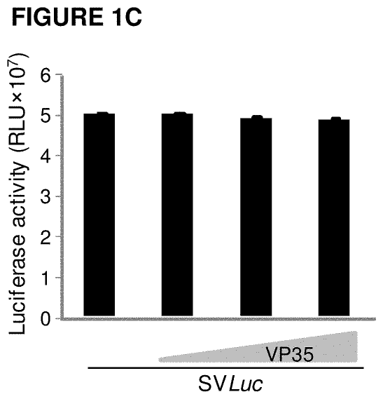

The capability of promoters to induce RNA interference in cells can be tested in an assay where an expression plasmid encoding a reporter protein under transcriptional control of a promoter to be tested is transferred to cells in the presence or absence of an expression plasmid encoding a viral suppressor of RNA interference, such as Ebola virus VP35 protein. In this assay, cells containing expression plasmids with promoters that induce RNA interference accumulate low amounts of reporter protein in the absence of a viral suppressor of RNA interference. Cells containing expression plasmids with promoters that do not induce RNA interference accumulate high amounts of reporter protein independent of the presence or absence of a viral suppressor of RNA interference (FIGS. 1A-1C).

Similar to the cytomegalovirus immediate early promoter and the retroviral long terminal repeat promoters the SV40 early promoter present in the expression plasmids described in U.S. 2010/0160415 and in the commercially available expression plasmids such as pGL3, pSG5, pRC/RSV are potent inducers of RNA interference in cells and thus of adaptive immune responses in vivo. The commercially available SV40 early promoters comprise only a part of the SV40 intergenic region including the domains with binding sites for transcription factors, the origin of replication and the transcriptional enhancer sequences. A number of reports name the potential use of unspecified SV40 promoters (WO 2003/045316 A2; WO 2006/124375 A2; US 2002/068715 A1; US 2012/076808 A1) for restoring immune tolerance in vivo. The unspecified SV40 promoters comprise part of the SV40 intergenic region and are not equivalent to the full-length SV40 early and late promoter of the present invention (SEQ ID NO: 19, SEQ ID NO: 2).

The present invention is therefore contra-intuitive for a skilled person. Moreover, it is known to a skilled person that the small T antigen is an effective inhibitor of RNAi. Since RNAi is known to serve as an antiviral mechanism, it would be expected that a decrease in the amount of intracellular small T antigen leads to an increase in the RNAi-based antiviral activity, resulting in a reduced production of virus particles. The inventors surprisingly found that the opposite is true. When the large T antigen is provided in trans, i.e. the cell line produces the large T antigen, wherein both the cell line and the polyomavirus strain lack a functional small T antigen, polyomavius particles are produced in high amounts. The difference between the present invention and the results of Gauchat et al. is that in the experiments described in Gauchat et al. a functional large T antigen is provided in cis, i.e. on the polyomaviral vector that replicates in the infected cell. This obviously leads to partial cell death and to a very inefficient viral vector production.

In one preferred aspect, the method of the present invention includes the use of the SV40 intergenic region comprised between nucleotides 5171 and 334 in the SV40 genome published under the reference sequence NC_001669 at US National Center for Biotechnology Information (NCBI) (SEQ ID NO: 19). This region contains the early and late promoter with the binding sites for transcription factors, the origin of replication, all transcriptional enhancer sequences, the origin-of-replication, the encapsidation signals and a DNA secondary structure that is responsible for specific and efficient binding of the host DNA-dependent RNA polymerase (F. Vogel et al., Gene 16:331-334, 1981). The inability to induce RNA interference in a mammalian cell makes a polyomaviral full-length early and late promoter ideally suited for inducing immune tolerance in vivo (see Example 15).

Since the full-length SV40 early and late promoter sequence also comprises the viral origin-of-replication and encapsidation signals, the expression plasmid was used for the production of replication-deficient or replication-defective SV40 gene delivery vector particles (denoted SV Luc). Human PBMCs transduced with SV Luc particles also secrete tolerogenic cytokines. In addition, the macrophages and dendritic cells present in the transduced mononuclear cells carried tolerogenic co-stimulatory molecules and chemokine receptors on their surface showed high levels of intracellular MHC, indicating that these cells remained immature. In contrast, human PBMCs transduced with a recombinant SV40 vector encoding the firefly luciferase under transcriptional control of the cytomegalovirus immediate early promoter, secreted pro-inflammatory cytokines. The macrophages and dendritic cells present in the transduced cells carried proinflammatory co-stimulatory molecules and chemokine receptors on their surface and showed low levels of intracellular MHC, indicating that these cells maturated. It was concluded from this surprising result that although the entire full-length SV40 intergenic region is of viral origin, it is capable of inducing immune tolerance. Since polyomaviral gene delivery vectors, including SV40 vectors, are immunologically inert in humans, the vectors have the unique capability of inducing an active immune tolerance response to a self-protein encoded by the vector. Replication-deficient or replication defective polyomaviral vectors, in which the early or late coding domain has been replaced with a gene encoding an auto-antigen and where the auto-antigen is expressed in transduced cells under transcriptional control of the entire full-length polyomaviral intergenic region, are highly efficient in restoring auto-antigen-specific immune tolerance in patients with a degenerative disease, such as an autoimmune disease.

SV40 belongs to the Polyomaviruses, which comprises a family of non-enveloped DNA (double-stranded) viruses with icosahedral capsids. SV40 has a circular genome with a length of 5.25 kilo base pairs and consists of two regulatory regions: the promoter/origin region and the polyadenylation region, as well as the early and late regions. The SV40 early promoter controls transcription of the early region, which encodes the small and large T antigens. The late promoter controls transcription of the late region, which encodes the viral capsid proteins (VP1, VP2 and VP3).

In polyomaviral vectors, T antigen-specific sequences have been deleted, not only for rendering the vectors replication-incompetent, but also to completely eliminate their immunogenicity. Polyomaviral vectors derived from SV40 have been produced and tested, in which the therapeutic genes or nucleic acids are expressed in target cells under transcriptional control of the viral early promoter. The vectors have been used for gene transfer into a wide variety of human tissues and cell types, for example, bone marrow (D. Rund et al., Human Gene Therapy 9:649-657, 1998), the liver (D. S. Strayer and M. A. Zern, Seminars in Liver Disease 19:71-81, 1999) and dendritic cells (M. Vera et al., Molecular Therapy 12:950-959, 2005).

Polyomaviral vectors, such as SV40, are known to infect non-dividing as well as dividing cells. Since SV40 is a macaque polyomavirus that does not occur in the human population and SV40 vectors are replication deficient, SV40 vectors are non-immunogenic in humans and allow for repeated administration to the same individual (D. S. Strayer and M. A. Zern, Seminars in Liver Disease 19:71-81, 1999).

The present invention relates to the use of a recombinant gene comprising a heterologous DNA encoding one or multiple auto-antigens or antigenic parts thereof and an entire full-length polyomaviral intergenic region, for restoring immune tolerance to the auto-antigens in vivo.

In the present invention, the heterologous DNA refers to a DNA sequence that codes for one or multiple auto-antigens or antigenic parts thereof. The term auto-antigen as used herein is used for a protein produced by cells of a patient suffering from a degenerative or dystrophic disease that is specifically targeted by the patients' adaptive immune system and that as a result is involved in the destruction of the cells in the course of the disease. A suitable example of a degenerative or dystrophic disease is an auto-immune disease.

The adaptive immune system comprises humoral and cellular components. The humoral adaptive immune system consists of antibodies produced by B lymphocytes. The cellular adaptive immune system mainly consists of T lymphocytes. Traditionally, the classification of a disease as autoimmune has been based on the detection of antibodies denoted autoantibodies that recognize and bind to auto-antigens.

Methods to identify auto-antigens that are involved in autoimmune tissue destruction are based on detection of components of the cellular adaptive immune system including but not limited to: i) construction of an expression cDNA library of the tissue affected by a degenerative or dystrophic disease; ii) expression of the cDNAs in human cells; iii) bring the human cells into contact with PBMCs obtained from patients with the degenerative or dystrophic disease; iv) measure the amounts of pro-inflammatory cytokines produced by activated lymphocytes present in the PBMC population. The cDNA molecules that induce the production of pro-inflammatory cytokines in PBMCs from patients with a degenerative or dystrophic disease, encode a major auto-antigen involved in the autoimmune tissue destruction.

An alternative method to identify auto-antigens that are involved in autoimmune tissue destruction is also based on detection of components of the cellular adaptive immune system. The alternative method includes: i) construction of an expression cDNA library of the tissue affected by a degenerative disease; ii) expression of the cDNAs in PBMCs obtained from patients with the degenerative disease; iii) measure the amounts of pro-inflammatory cytokines produced by activated T lymphocytes present in the PBMC population. The cDNA molecules that induce the production of pro-inflammatory cytokines in PBMCs from patients with a degenerative disease, encode a major auto-antigen involved in the autoimmune tissue destruction.

In order to further confirm that the identified auto-antigen is a major driver of autoimmune destruction, the protein encoded by the cDNA is produced in cells using standard techniques well known in the art. Purified protein is subsequently injected into a mammal together with a strong adjuvant such as Freund's complete adjuvant. The injected mammal will subsequently be monitored for the development of degenerative or dystrophic disease symptoms. The mammals that develop degenerative or dystrophic disease symptoms have been administered with a major auto-antigen involved in the autoimmune cell destruction.

A cell line according to the invention may be derived from any suitable cell line known in the art such as MDCK, PER.C6, HEK293, CV1 and the like, but is preferably a Vero or CHO cell line.

A suitable cell line according to the invention is a polyomavirus permissive cell line incapable of expressing the polyomaviral small T antigen and preferably comprises the following genetic elements:
i) the polyomaviral large T antigen coding domain or part thereof, and optionally
ii) a selectable marker such as a neomycin resistance gene, puromycin resistance gene, hygromycin resistance gene or other marker.

Such a cell line may be devoid of the large intron of the polyomavirus early transcript harbouring small T antigen-specific DNA sequences

The cell line in a preferred embodiment may include a transcriptional enhancer sequence stably integrated into the chromosomal DNA of the cell line, such that it may be further selected on the basis of the activity of the transcriptional enhancer. Such markers and such selection procedures are well known in the art.

Different polyomaviral vector production cell lines, eg SV40 viral vector producer cell lines, may be generated by transfecting the cells with different vectors, such as plasmids, depending on their pedigree. The methodology for transfection of cell lines is well known in the art. For example, the Vero cell line is widely used for the production of virus particles for vaccines. This has found many applications in prophylaxis of viral diseases.

A suitable cell line may be obtained by transfection with a first plasmid comprising the following components
i) the polyomaviral large T antigen coding domain or part thereof, devoid of the large intron of the polyomavirus early transcript harbouring small T antigen-specific sequences and optionally
ii) a selectable marker such as a neomycin resistance gene, puromycin resistance gene, hygromycin resistance gene or other marker.

Thus a single vector or plasmid could carry both the polyomaviral large T antigen coding domain and a selectable marker sequence. It is also possible that two separate DNA carrying vectors or plasmids are utilized, one carrying the polyomaviral large T antigen coding domain and a second carrying the selectable marker, depending on design. Any further genetic elements that may be needed to confer a polyomaviral vector production capability on a producer cell line may also be placed onto one or more DNA vectors or plasmids that may then be used to transfect the production cell line of choice. For instance, the Vero production cell line does not already contain polyomaviral T antigen sequences in it, so the large T antigen coding domain may be added into it, and the resulting nascent producer cells harbouring the large T antigen coding domain in them may then be selected for, by using a selectable marker system.

For a number of autoimmune diseases the major auto-antigens involved in the autoimmune cell destruction are known. For the majority of degenerative or dystrophic diseases however, the auto-antigens await to be identified using the confirmatory methods described above or using other immunological methods known to those skilled in the art. Examples of auto-antigens involved in autoimmune disease are: the neurofilament light (NFL) chain and alpha-actinin involved in amyotrophic lateral sclerosis; forminotransferase cyclodeaminase involved in autoimmune hepatitis; tissue transglutaminase involved in celiac disease; collagen type IV involved in Goodpasture' s syndrome; thyroglobulin involved in Hashimoto's thyroiditis; platelet-specific glycoproteins IIb/IIIa, Ib/IX, Ia/IIa and IV involved in idiopathic thrombocytopenic purpura; endothelial cell growth factor involved in Lyme disease; the acetylcholine receptor involved in myasthenia gravis; hypocretin-1/orexin involved in varcolepsy; aquaporin 4 involved in neuromyelitis optica; RNA-induced silencing complex (RISC) components involved in systemic lupus erythematosus; myelin oligodendrocyte glycoprotein (MOG) and myelin basic protein (MBP) involved in multiple sclerosis; golgi SNAP receptor complex member 1 (GOSR1) involved in obesity and obesity-induced insulin resistance (type 2 diabetes); keratin involved in psoriasis; type II collagen involved in Rheumatoid arthritis; proinsulin and glutamic acid decarboxylase 65 involved in type 1 diabetes; elastin and beta-2-glycoprotein I involved in atherosclerosis and chronic obstructive pulmonary disease; alpha-fodrin involved in Sjogren's syndrome; the glutamate receptor type 3 (GluR3) involved in epilepsy; histidyl-tRNA-synthetase and myosin-binding C protein in muscular dystrophies; cochlin and beta-tectorin in Meniere disease; arrestin, type I collagen and interphotoreceptor retinoid-binding protein in ophthalmological dystrophies and autoimmune diseases.

The polyomavirus intergenic region sequence resides between the early and late coding regions and comprises the full-length polyomaviral early and late promoter. In a preferred embodiment, the invention relates to the use of the SV40 intergenic region on the SV40 genomic DNA residing between the early and late coding regions and comprising the full-length SV40 early and late promoter (SEQ ID NO: 19 and SEQ ID NO: 2 comprise the full-length early and late SV40 promoter).

By “promoter” is meant a sequence of nucleotides from which transcription may be initiated of DNA operably linked downstream (i.e., in the 3′ direction on the sense strand of double-stranded DNA). “Operably linked” means joined as part of the same nucleic acid molecule, suitably positioned and oriented for transcription to be initiated from the promoter. DNA operably linked to a promoter is “under transcriptional control” of the promoter.

Generally speaking, those skilled in the art are well able to construct a recombinant gene comprising a heterologous DNA encoding one or multiple auto-antigens or antigenic parts thereof and full-length polyomaviral early and late promoters. For further details see, for example, Molecular Cloning: a Laboratory Manual 2nd edition, Sambrook et al., Cold Spring Harbor Laboratory Press, 1989.

In another preferred embodiment the recombinant gene of the invention is packaged in recombinant polyomaviral gene delivery vector particles, encoding one or multiple auto-antigens or antigenic parts thereof under transcriptional control of the full-length polyomaviral early and late promoter, for restoring immune tolerance to the antigens in vivo.

In another preferred embodiment the recombinant gene of the invention is packaged in recombinant primate polyomaviral gene delivery vector particles, such as a simian polyomavirus, more in particular an SV40, Simian virus 12 (SV12), Lymphotropic polyomavirus, African green monkey polyomavirus or Chimpanzee polyomavirus gene delivery vector particles.

In a preferred embodiment, the preparation according to the invention relates to a composition comprising a primate polyomavirus, such as a simian polyomavirus, more in particular an SV40, Simian virus 12 (SV12), Lymphotropic polyomavirus, African green monkey polyomavirus or Chimpanzee polyomavirus. Cell lines permissive for the primate polyomavirus are preferably selected from the group consisting of Vero cells, CV1 cells, PerC.6 cells, HEK293 cells and the like.

In another embodiment, the preparation according to the invention relates to a composition comprising a rodent polyomavirus such as mouse or hamster polyomavirus, more in particular a Murine polyoma virus or Hamster polyomavirus. Cell lines permissive for the mouse or hamster polyomavirus are preferably selected from the group consisting of CHO cells and the like.

The present invention also discloses the generation of vector production cell lines for the production of recombinant polyoma viral vector particles that are safe for medical use.

Accordingly, the invention relates to a cell line permissive for a polyomavirus, said cell line being capable of expressing a functional large T antigen and incapable of expressing a small T antigen. Such a cell line adequately supports the safe production of recombinant polyomaviral vector particles without the risk of obtaining wild type revertant polyomavirus particles since the cells lack any homologous sequences between the chromosomal DNA of the cell and the recombinant polyomavirus DNA. The gene encoding the large T antigen is preferably stably integrated in the genome of the cell.

The term “recombinant polyomaviral vector” in this context is to be interpreted as a polyomavirus incapable of expressing a functional polyomaviral large T antigen, preferably incapable of expressing a functional large and small T antigen. Such a recombinant viral vector may for instance lack the coding sequence for either the large T antigen or both the large T and the small T antigen.

The expression “permissive for a polyomavirus” in this context means that that the cell line supports the replication of polyomavirus particles upon infection with the polyomavirus or upon the introduction of polyomavirus DNA by transfection or other means of delivering DNA into a cell.

In another aspect, the invention provides a method for producing recombinant polyomavirus particles incapable of expressing a functional small T antigen comprising the steps of
a. a. Providing a cell line permissive for a wiidtype polyomavirus, said cell line being capable of expressing a functional polyomaviral large T antigen and incapable of expressing a functional small antigen,
b. b. Introducing into said cell line a polyomavirus DNA incapable of encoding a functional small T antigen,
c. c. Culturing said cells in a growth medium under conditions allowing the formation of polyomavirus particles and
d. d. Harvesting the recombinant polyomavirus particles from the cell culture.

Immune tolerance can be induced in a subject by ectopic expression of the recombinant gene of the invention, or by recombinant polyomaviral gene delivery vector particles of the invention. The term ectopic expression used herein refers to the accumulation of one or multiple auto-antigens or antigenic parts thereof in cell types that under normal conditions do not accumulate the proteins or antigenic parts thereof. Induction of immune tolerance can be experimentally demonstrated as follows: i) human PBMCs are transfected with a recombinant gene of the invention, or transduced with recombinant primate polyomaviral gene delivery vector particles of the invention, encoding one or multiple auto-antigens or antigenic parts thereof; ii) the transfected or transduced PBMCs secrete tolerogenic cytokines such as IL10 and transforming growth factor beta, whereas the amounts of proinflammatory cytokines such as IL3, IL4, ILS, IL6, IL12, IL13, IL14, interferon alpha, interferon beta, interferon gamma and tumor necrosis factor alpha remain low; iii) the macrophages and dendritic cells isolated from the transduced PBMCs remain immature. The maturation stage of the macrophages and dendritic cells can be estimated by measuring the amounts and localization of specific maturation markers such as B7-1, B7-2, CD40, CD25, CD58, CD83, Fc receptors and intercellular adhesion molecule-1.

The invention now permits for the first time the preparation of compositions comprising recombinant polyomaviral vectors in sufficient amounts for therapeutic purposes, without the risk of contamination with wildtype polyomaviruses that occur from recombination between polyomaviral vector DNA and host cell DNA. This phenomenon is well described in the literature (Gluzman Y., Cell 23: 175-182, 1981; Oppenheim A. and Peleg A., Gene 77: 79-86, 1989; Vera M. et al., Molecular Therapy 10: 780-791, 2004).

The frequency with which this recombination occurs is less well documented however. The estimates vary greatly. Shaul et al estimated that recombination could occur with a frequency of at least 10⁻⁶ (Shaul et al., Proc. Natl. Acad. Sci. USA 82: 3781-3784, 1985), whereas more recent estimates show a much higher recombination rate, in the order of 10⁻³ (Arad et al., Virology 304: 155-159, 2002).

A complicating factor in estimating the frequency of recombination is that the wildtype polyomavirus replicates faster than the recombinant polyomaviral vector lacking the genes encoding functional T antigens.

Therefore, we established the maximum number of recombinant SV40 vector particles that could be produced in a conventional cell culture according to the prior art, without the appearance of any wild type revertants.

Therefore, we infected COS-1 cells with recombinant SV40 vector particles according to a standard protocol (Vera M. et al., Molecular Therapy 10: 780-791, 2004) and calculated the maximum number of viral vector particles that could be produced without the occurrence of a single detectable genome of the wildtype virus as detected by a very sensitive quantitive PCR assay.

It was found that up to 1×10⁴ viral vector particles could safely be produced without the occurrence of a detectable amount of wildtype revertants in the majority of experiments performed. A significant number of preparations comprising 1×10⁵ viral vector particles however, was positive for wildtype revertants, whereas all of the preparations comprising 1×10⁶viral vector particles were contaminated with wildtype viruses and thus unsafe for medical use.

The fact that the polyomaviral vector preparations according to the prior art are unsafe for medical use is underlined by the fact that the wildtype SV40 particles in the preparations comprising more than 1×10⁶recombinant SV40 vector particles were able to infect SV40-permissive cells in vitro when tested according to a method of the prior art as described by Katzman R. B. et al., (Journal of Virological Methods 150: 7-13, 2008).

The invention now allows for the first time the preparation of a composition comprising more than 1×10⁶polyomaviral vector particles without any wildtype polyomavirus particles being present in the composition. The invention therefore relates to composition comprising more than 1×10⁶polyomaviral vector particles incapable of expressing a functional polyomaviral small T antigen and incapable of replicating in cells which are permissive for the wildtype polyomavirus. Such preparations may advantageously contain 1×10⁷ vector particles or more, such as up to 1×10⁸, 1×10⁹, 1×10¹⁰, or 1×10¹¹ vector particles or more.

The expression “incapable of replicating in cells which are permissive for the wildtype polyomavirus” means that the composition does not contain a single wildtype revertant polyomavirus particle among the at least 1×10⁶polyomaviral vector particles. This may be measured either by the quantitative PCR assay as described by Vera M. et al., Molecular Therapy 10: 780-791, 2004, or by infecting a cell line permissive for the wildtype polyomavirus present in the composition. In the latter case the absence of a single plaque in the plaque assay (Katzman R. B. et al., Journal of Virological Methods 150: 7-13, 2008) indicates the absence of a single wildtype polyomavirus particle.

In another preferred embodiment, the invention relates to the use of the recombinant genes or polyomaviral gene delivery vector particles to restore immune tolerance in vivo. Restoration of immune tolerance can be experimentally demonstrated as follows: animal models of autoimmune disease are administered with a recombinant gene or vector of the invention, encoding one or multiple auto-antigens or antigenic parts thereof that are involved in the autoimmune cell destruction in the animal models. As a result the immune tolerance to the auto-antigens is restored and the progression of the autoimmune disease symptoms in the treated animals stops.

In further aspects of the invention there is provided a pharmaceutical composition for the treatment of an individual suffering from a disease. The pharmaceutical composition may comprise a therapeutically effective amount of one or more polyomavirus vectors such as SV40, prepared according to a process of the invention and a pharmaceutically acceptable carrier or diluent. The pharmaceutical compositions of the invention can be formulated in any suitable form for administration to the individual in need thereof. Such formulations may be in any form for administration such as topical, oral, parenteral, intranasal, intravenous, intramuscular, intralymphatic, subcutaneous, intraocular or even transdermal administration.

The pharmaceutical compositions of the invention generally comprise a buffering agent, an agent that adjusts the osmolarity thereof, an optionally, one or more pharmaceutically acceptable carriers, excipients and/or additives as known in the art. Supplementary active ingredients may also be incorporated into the compositions of the invention. The carrier may be a solvent or dispersion medium containing, for example, water, ethanol, polyol (for example, glycerol, propylene glycol, and liquid polyethylene glycol, and the like), suitable mixtures thereof, and vegetable oils. The correct fluidity may be maintained by the use of a coating, such as lecithin, by the maintenance of the required particle size in the case of dispersion and by the use of surfactants.

The invention also relates to compositions comprising recombinant genes or polyomaviral vectors and uses thereof as treatment for autoimmune diseases. The pharmaceutical composition may comprise a therapeutically effective amount of one or more recombinant genes, or one or more polyomaviral vectors such as SV40 vectors, and a pharmaceutically acceptable carrier or diluent. The pharmaceutical compositions of the invention can be formulated in any suitable form for administration to the individual in need thereof. Such formulations may be in any form for administration such as topical, oral, parenteral, intranasal, intraocular, intravenous, intramuscular, intralymphatic, subcutaneous or transdermal administration.

The pharmaceutical compositions of the invention generally comprise a buffering agent, an agent that adjusts the osmolarity thereof, and optionally, one or more pharmaceutically acceptable carriers, excipients and/or additives as known in the art. Supplementary active ingredients may also be incorporated into the compositions of the invention. The carrier may be a solvent or dispersion medium containing, for example, water, ethanol, polyol (for example, glycerol, propylene glycol, liquid polyethylene glycol, and the like), suitable mixtures thereof, and vegetable oils. The correct fluidity may be maintained by the use of a coating, such as lecithin, by the maintenance of the required particle size in the case of dispersion and by the use of surfactants.

In a preferred embodiment, the polyomavirus DNA is introduced into the cell by transfection of the DNA.

In summary, the invention provides the following:

A vector comprising a nucleic acid sequence encoding an antigen under the transcriptional control of a polyomaviral early and/or late promoter, for use in inducing an immune tolerance towards the antigen in a host organism. This may also be stated as that the method according to the invention provides a method for inducing an immune tolerance towards the antigen in a host organism, wherein a vector is administered to the host organism, wherein the vector comprises a nucleic acid sequence encoding an antigen under the transcriptional control of a full-length polyomaviral early and late promoter.

In a preferred embodiment the antigen is under the control of a full-length polyomaviral early and late promoter, such as the full-length SV40 early and late promoter according to SEQ ID NO: 19 or SEQ ID NO: 2.

Advantageously, the antigen in the above mentioned method is an auto-antigen. This allows for the treatment of a number of auto-immune diseases. Advantageously, the auto-antigen is selected from the group consisting of the neurofilament light (NFL) chain, alpha-actinin, beta-2 glycoprotein I, forminotransferase cyclodeaminase, tissue transglutaminase, type IV collagen, thyroglobulin, platelet-specific glycoproteins IIb/IIIa, Ib/IX, Ia/IIa and IV, endothelial cell growth factor, the acetylcholine receptor, aquaporin 4, RNA-induced silencing complex (RISC) components, myelin oligodendrocyte glycoprotein, myelin basic protein, the golgi SNAP receptor complex member 1 (GOSR1), keratin, type II collagen, proinsulin and glutamic acid decarboxylase 65, elastin, alpha-fodrin, the glutamate receptor type 3 (GluR3), histidyl-tRNA-synthetase, myosin-binding C protein, cochlin, beta-tectorin, arrestin, type I collagen and interphotoreceptor retinoid-binding protein.

The term “antigen” may relate to any substance (as an immunogen or a hapten) that is capable of eliciting an immune response either alone or after forming a complex with a larger molecule for instance a protein and that is capable of binding with a product (as an antibody or T cell) of the immune response. In a preferred embodiment, the antigen is an auto-antigen. The term “auto-antigen” is used herein to indicate an antigen derived from the body itself as opposed to non-self antigens which are foreign to the body in which they elicit an immune response. The term “antigen” or “auto-antigen” should not be so narrowly construed as that it is required to consist of a full-length antigen such as a full-length protein, glycoprotein or polysaccharide. It suffices when the antigen consists of an antigenic part or an immunogenic part of the full-length auto-antigen. For instance, if only part of a full-length antigen or auto-antigen is used, it suffices when the part comprises an epitope against which the immune tolerance is to be induced. For general purposes, a fragment of at least 6 amino acids, such as 7, 8, 9, 10, 11, 12, 13, 14, 15 or more amino acids suffices. More preferred is the use of longer fragments, most preferred is the use of the full-length antigen comprising many if not all epitopes.

In another embodiment, the auto-antigen is hypocretin-1 or orexin.

Methods according to the invention may be advantageously employed in the treatment of an auto-immune disease selected from the group consisting of neurological diseases, Alzheimer's dementia, amyotrophic lateral sclerosis, bipolar disorder, depression, epilepsy, Lyme disease, multiple system atrophy, multiple sclerosis, myasthenia gravis, narcolepsy, neuromyelitis optica, Parkinson's disease, cchizophrenia, metabolic diseases, diabetes, type 2 diabetes, Hashimoto's thyroiditis, obesity, ophthalmological diseases, age-related macular degeneration, glaucoma, uveitis, gastro-enteric diseases, inflammatory bowel diseases, Crohn's disease, ulcerative colitis, dermatological diseases, psoriasis, scleroderma, vitiligo, cardiovascular diseases, atherosclerosis, cardiomyopathy, Coxsackie myocarditis, rheumatoid arthritis and rheumatic fever.

Additionally, the auto-immune disease may be selected from type-1 diabetes, retinitis pigmentosa, Leber's congenital amaurosis, Stargardt macular dystrophy, achromatopsia, retinoschisis and citelliform macular dystrophy, orthopedic diseases, lupus erythematosus, muscular diseases, polymyositis, dermatomyositis, inclusion body myositis, Duchenne muscular dystrophy, Becker muscular dystrophy, Miyoshi myopathy, limb-girdle muscular dystrophy, congenital muscular dystrophy, distal muscular dystrophy, Emery-Dreyfuss muscular dystrophy, fascio-scapulohumeral muscular dystrophy, myotonic muscular dystrophy and oculopharyngeal muscular dystrophy.

In yet another method according to the invention, the host is human.

A particularly advantageous use includes administering to the host a vector comprising a full-length polyomaviral early and late promoter and a nucleic acid encoding an antigen wherein the expression of the antigen is under the transcriptional control of the full-length polyomaviral early and late promoter according to SEQ ID NO: 19 or SEQ ID NO: 2.

Vectors as described herein may be administered intravenously. Particularly preferred is a replication-defective SV40 vector.

The invention will now be further described with reference to the following examples:

### EXAMPLES

### Example 1

### Construction of the SV40 Derived Gene Delivery Vector

Six oligonucleotides were designed:

WdV101:
(SEQ ID No. 2)
CCGCTCGAGTTGCGGCCGCTGTGCCTTCTAGTTGCCAGCCATC

(containing a Xhol and a Notl restriction site) and

WdV102: GGTACCATAGAGCCCACCGCATCCCCAGCATGCC (SEQ ID No. 3) (containing a Kpnl restriction site) and

WdV103: GGCCGCTTTATTAATTAAGCCCTGCAGGTTGTTTAAACTTGGCGC GCCTTAT (SEQ ID. No. 4) (contains from 5′ to 3′ subsequently a Notl sticky restriction site, a Padl, SbfI, Pmel and an AscI intact restriction site and a ClaI sticky restriction site) and

WdV104: CGATAAGGCGCGCCAAGTTTAAACAACCTGCAGGGCTTAATTAAT AAAGC (SEQ ID No. 5) (contains from 3′ to 5′ subsequently a Notl sticky restriction site, a PadI, SbfI, PmeI and an AscI intact restriction site and a ClaI sticky restriction site) and

WdV105:
(SEQ ID NO. 6)
CGGGATCCAGACATGATAAGATACATTG

(containing a BamHI restriction site) and

WdV106:
(SEQ ID No. 13)
ATAGTTTAGCGGCCGCAACTTGTTTATTGCAGCTTATAATGG

(containing a Notl restriction site).

Purified plasmid DNA of the SV40 vector pSL-PL (De La Luna, S. et al., Journal of General Virology 74: 535-539, 1993) was subjected to PCR using oligonucleotides WdV105 and WdV106. The resulting amplified DNA fragment comprised the SV40-polyadenylation signal flanked by a BamHI restriction site at the 5′ end and a Notl restriction site at the 3′ end. This SV40 polyadenylation signal fragment was digested with BamHI and NotI and the resulting 150 bp long DNA fragment was isolated from an agarose gel and cloned into a likewise digested pBluescript SK-plasmid (Promega), yielding pAM002.

Purified pEF5/FRT/5-DEST (Invitrogen) plasmid DNA was subjected to PCR using oligonucleotides WdV101 and WdV102. The resulting amplified DNA fragment comprising the bovine growth hormone (BGH) polyadenylation signal flanked by subsequently a Xhol and a Notl restriction site at the 5′ end and an Kpnl restriction site at the 3′ end. This BGH polyadenylation signal fragment was digested with Kpnl and Notl, and the resulting 250 bp long DNA fragment was isolated from an agarose gel and ligated into the likewise digested pAM002 plasmid. Transformation with this ligation mixture was performed in a methylation insensitive E. coli strain. This resulted in plasmid pAM003.

The two complementary oligonucleotides WdV103 and WdV104 were annealed by incubating them in a water bath that was cooling down autonomously from boiling temperature to room temperature, yielding a DNA linker containing subsequently a Notl sticky restriction site, a PadI, SbfI, PmeI and a Ascl intact restriction site and a ClaI sticky restriction site. This linker was ligated into the pAM002 plasmid that was digested with NotI and ClaI and isolated from an agarose gel. The ligation mixture was subsequently used to transform a methylation insensitive E. coli strain, yielding pAM004.

Purified plasmid DNA of the SV40 vector pSL-PL was digested with ClaI and BamHI. The resulting 2.6 kb DNA fragment that contains the SV40 origin and the SV40 late region is purified from agarose and cloned into likewise digested pAM004. This resulted in the new SV40 vector plasmid pAM005.

### Example 2

### Construction of a Replication Deficient SV40 Destination Vector (pSVac-dest).

Eight oligonucleotides were designed:

WdV101:CCGCTCGAGTTGCGGCCGCTGTGCCTTCTAGTTGCCAGCCA TC (SEQ ID NO: 2) (containing a Xhol and a Notl restriction site) and WdV102: GGTACCATAGAGCCCACCGCATCCCCAGCATGCC (SEQ ID NO: 3) (containing a Kpnl restriction site) and WdV103: GGCCGCTTTATTAATTAAGCCCTGCAGGTTGTTTAAACTT GGCGCGCCTTAT (SEQ ID. NO: 4) (contains from 5′ to 3′ sequentially a Notl sticky restriction site, a PacI, Sbfl, Pmel and an Ascl intact restriction site and a CIaI sticky restriction site) and WdV104: CGATAAGGCGCGCCAAGTTTAAACAACCTGCAGGGCTTAATT AATAAAGC (SEQ ID NO: 5) (contains from 3′ to 5′ sequentially a Notl sticky restriction site, a PadI, Sbfl, Pmel and an Ascl intact restriction site and a CIaI sticky restriction site) and WdV105: CGGGATCCAGACATGATAAGATACATTG (SEQ ID NO: 6) (containing a BamHI restriction site) and WdV106: ATAGTTTAGCGGCCGCAATGAATGCAATTGTTGTT GTTAACTTG (SEQ ID NO: 13) (containing a Notl restriction site) and WdV108: TGGCGCGCCTATAGGGAGACCCAAGCTGGCTAG (SEQ ID NO: 8) (containing an Ascl restriction site) and WdV109 CAATCATACCGTTTAAACGAACCGCGGGCCCTCTAGAC (SEQ ID NO: 9) (containing a PmeI restriction site).

Purified plasmid DNA of the SV40 vector pSL-PL (S. De La Luna et al., Journal of General Virology 74:535-539, 1993) was subjected to PCR using oligonucleotides WdV105 and WdV106. The resulting amplified DNA fragment comprised the SV40-polyadenylation signal flanked by a B amHI restriction site at the 5′ end and a Notl restriction site at the 3′ end. This SV40 polyadenylation signal fragment was digested with BamHI and Notl and the resulting 150 base pairs long DNA fragment was isolated from an agarose gel and cloned into a likewise digested pBluescript SK-plasmid (Stratagene), yielding pAM002.

Purified pEF5/FRT/5-DEST (Invitrogen) plasmid DNA was subjected to PCR using oligonucleotides WdV101 and WdV102. The resulting amplified DNA fragment comprising the bovine growth hormone (BGH) polyadenylation signal flanked by sequentially a Xhol and a Notl restriction site at the 5′ end and a Kpnl restriction site at the 3′ end. This BGH polyadenylation signal fragment was digested with Kpnl and Notl, and the resulting 250 base pairs long DNA fragment was isolated from an agarose gel and ligated into the likewise digested pAM002 plasmid. Transformation with this ligation mixture was performed in a methylation insensitive E. coli strain. This resulted in plasmid pAM003.

The two complementary oligonucleotides WdV103 and WdV104 were annealed by incubating them in a water bath that was cooling down autonomously from boiling temperature to room temperature, yielding a DNA linker containing sequentially a Notl sticky restriction site, a PacI, Sbfl, Pmel and an Ascl intact restriction sites and a CIaI sticky restriction site. This linker was ligated into the pAM003 plasmid that was digested with Notl and CIaI and isolated from an agarose gel. The ligation mixture was subsequently used to transform a methylation insensitive E. coli strain, yielding pAM004.

Purified plasmid DNA of the SV40 vector pSL-PL was digested with CIaI and BamHI. The resulting 2.6 kilo base pairs DNA fragment that contains the SV40 origin and the SV40 late region is purified from agarose and cloned into likewise digested pAM004. This resulted in the new SV40 vector plasmid pAM005.

Purified pEF5/FRT/5-DEST (Invitrogen) plasmid DNA was subjected to PCR using oligonucleotides WdV108 and WdV109. The resulting amplified DNA fragment comprises the GATEWAY® recombination cassette including the ccdB gene and a chloramphenicol resistance gene flanked by AttR1 and AttR2 recombination sequences. These in turn are flanked by an Ascl restriction site at the 5′ end and a Pmel restriction site at the 3′ end. This fragment was subsequently digested with Ascl and Pmel, and the resulting 1844 base pairs long DNA fragment was isolated from an agarose gel and ligated into the likewise digested pAM005 plasmid, resulting in pAM006 (pSVac-dest).

Correct cloning of the cassette was confirmed by sequencing. Functionality was confirmed by successful recombination of transgenes and by showing that the Chloramphenicol-resistance and the ccdB gene in the GATEWAY® cassette were functional.

### Example 3

### Molecular Cloning of an SV40 Encoding Luciferase Expression Vector (SVLuc)

The expression plasmid pGL3 (Promega) was used as template for cloning of the firefly luciferase (Photinus pyralis) into pSVac destination vector by PCR. Two oligonucleotides were designed:

WdV-070: GGGGACAAGTTTGTACAAAAAAGCAGGCTATGGAAGAC GCCAAAAACATAAAGAAAGGC (SEQ ID NO: 10) and

WdV-071: GGGGACCACTTTGTACAAGAAAGCTGGGTTTACACGGCGA TCTTTCCGCCCTTC (SEQ ID NO: 11) containing, respectively, AttB1 and AttB2 recombination sequences.

Purified pGL3 plasmid DNA was subjected to PCR using oligonucleotides WdV070 and WdV071. The purified PCR product was subsequently recombined by a BP reaction in pDONR221 (Invitrogen) to create a luciferase entry clone, pAM007. This entry clone was used for a GATEWAY® LR reaction to recombine the firefly luciferase coding sequence into pSVac-dest (pAM006), resulting in the pSVLuc vector plasmid (pAM008).

### Example 2

### Molecular Cloning of a SV40 Luciferase Expression Vector and the Production of Recombinant SV40 Luciferase Vector Particles

The expression plasmid pGL3 (Promega) was used as template for cloning of the firefly luciferase using PCR. Two oligonucleotides were designed WdV389: 5′-TTGGCGCGCCATGGAAGACGCCAAAAACATAAAGAAAGGC-3′ (SEQ ID NO: 14) and WdV407: 5′-CCCTTAATTAATTACACGGCGATCTTTCCGCCCTTC-3′ (SEQ ID NO: 15) containing respectively restriction sites Ascl and PacI. The PCR amplified luciferase fragment was subsequently Ascl and PacI digested and ligated into pAM005, resulting in pAM006.

Two oligonucleotides were designed WdV437 5′ GGGATCCAGACATGATAAGATACATTG 3′ (SEQ ID NO: 16) and WdV442: ATAGTTTAGCGGCCGCAATGAATGCAATTGTTGTTGTTAACTTG (SEQ ID NO: 17) containing respectively BamHI and Notl restriction site. The pSL-PL vector was used as template for cloning of the large T antigen trailer sequence using PCR. The resulting PCR fragment was digested with BamHI and Notl and cloned into the BamHI and Notl (partially digested) pAM006, resulting in pAM020.

Recombinant SV40 vector particles encoding the firefly luciferase (SV-Luc) were produced according Vera M. et al., Molecular Therapy 10: 780-791, 2004. COS-1 cells were transfected with Notl-digested and recircularized pAM020 DNA and three days after transfection crude lysates were prepared from the cell culture by repeated freeze-thawing. The SV-Luc vector particles were amplified in one round in COS-1 cells growing in a T175 flask. The SV-Luc vector particles were finally concentrated and purified from the crude lysate by sucrose gradient ultracentrifugation, yielding a vector stock with 5×10¹¹ SV-Luc genome copies per milliliter cell culture.

### Example 4

### Molecular Cloning of an SV40 Encoding Myelin Oligodendrocyte Glycoprotein Expression Vector (SVMOG)

The coding DNA sequence for the human myelin oligodendrocyte glycoprotein (MOG, NCBI gene ID 4340) flanked by AttB1 and AttB2 recombination sites was synthesized chemically (GENEART®). The DNA fragment was subsequently recombined by a BP reaction in pDONR221 to create a MOG entry clone, pAM009. This entry clone was used for a GATEWAY® LR reaction to recombine the MOG coding sequence into pSVac-dest (pAM006), resulting in the pSVMOG vector plasmid (pAM010).

### Example 5

### Production and Purification of SV Luc and SVMOG Particles

Recombinant SV40 vector DNA encoding either the human MOG protein and firefly luciferase (pSVMOG and pSVLuc) were Notl digested to remove the bacterial backbone. The SV40 vector DNA was isolated from agarose gel and purified. Subsequently, the SV40 vector sequence was re-circularized using T4 ligase and used to transfect SuperVero cells growing in roller bottles (growth area: 850 squared centimeter; Greiner Bio One) at 20-70 percent confluence in OptiPRO™ serum-free media containing L-Glutamine 200 millimolar at 37 degrees Celsius and 5 percent carbon dioxide. SuperVero is a genetically engineered packaging cell line that was generated by the introduction of polyomaviral SV40 large T antigen into Vero cells.

SuperVero cells support the replication of replication-deficient recombinant SV40 (rSV40) vectors as described in detail in patent application WO/2010/122094.

Three and six days post transfection, the culture medium containing the vector particles was collected and replaced by fresh media. The pooled culture media containing SVMOG or SV Luc were purified using a dual membrane filter (1/0.5 micrometer Polysep II filter, MILLIPORE®) and 10 times concentrated by ultrafiltration/diafiltration (UFDF), a tangential flow filtration performed using a 300 kilodalton molecular weight cut-off membrane (Pall Corporation). Finally, the vector material was filtered through a 0.45 micrometer ACRODISC® filter (Pall Corporation), aliquoted and stored at 4 degrees Celsius.

To further increase the amount of vector particles pooled the vector was used for (at least) two subsequent transduction rounds. In each transduction round, SuperVero cells were transduced with 400 SVMOG or SVLuc vector genomes per cell. Three and six days post transduction, the rSV40 particle-containing culture medium was collected and replaced by fresh media. After each amplification round, the pooled culture medium was purified and concentrated as described above.

A third transduction round was performed on at least 40 roller bottles (growth area: 850 squared centimeter) containing 20-70 percent confluent SuperVero cells transduced with 400 SVMOG or SVLuc vector genomes per cell. Again, at three and six days post transduction the rSV40 vector particles containing culture medium was collected and replaced by fresh media. The collected culture media results in a final batch volume of at least 20 liter.

To remove unwanted nucleic acids (mainly in the form of host cell DNA), Benzonase was added (10 Units/milliliter, VWR International) in the presence of 2 millimolar MgC12 and incubated for 1-4 hours at room temperature. The material was subsequently clarified using a dual membrane filter (1/0.5 micrometer Polysep II filter) in order to remove the cell debris. After the clarification, the material was concentrated 10-60 fold using an UFDF step, followed by filtration through a 0.45 micrometer MILLIPAK® membrane (MILLIPORE®).

Remaining impurities were then removed from the vector material by a group separation chromatography using an AKTA explorer, 2.5 liter, 40 centimeters SEPHAROSE® 6 fast flow resin column (GE Healthcare Life Science). Ultraviolet (UV) absorbance at 260 and 280 nanometers was used for detection of vector particles and fractions containing the vector peak were mixed and stored overnight at 4 degrees Celsius. The column eluate pool containing SVMOG or SVLuc vector particles was concentrated to approximately 15 milliliter and the buffer was exchanged 10-fold by UFDF. Sucrose (5 percent volume/volume) and tween-80 (0.005 percent volume/volume) excipients were added and the final concentrated bulk product was sterile filtered (0.8/0.2 micrometer ACRODISC® PF filter, Pall Corporation), aliquoted in glass vials and stored at 4 degrees Celsius until use.

### Example 6

### Generation of a Vero Producer Cell Line and Production of Recombinant SV40 Vector Particles

Vero cells (Sigma-Aldrich order number: 88020401) were propagated and adapted to serum free culture DMEM medium (Invitrogen, product code: 41966-052). Adaptation to serum free conditions was performed by gradually reducing fetal bovine serum from 8, 6, 4, 2 and 0 percent in the medium each passage. From then the Vero-Serum Free (Vero-SF) cells were cultured in OptiPro SFM medium (Invitrogen) containing 2 percent L-glutamine at 37 degrees Celsius and 5 percent CO₂.

Vero-SF cells were transfected with pAM001 DNA using the transfection agent Exgen 500 (Fermentas, product code: R0511) according to the supplier's prescriptions. The transfected Vero-SF cells were subsequently selected for integration of the SV40 large T expression gene cassette into the chromosomal DNA by adding 2 μg/ml puromycine to the cell culture medium. Surviving colonies were isolated and propagated in OptiPro SFM medium containing 2 μg/ml puromycine and 2 percent L-glutamine. Puromycin-resistant cells were transferred OptiPro SFM medium containing 2 percent L-glutamine and 10 percent DMSO and stored at −156 degrees Celsius.

### Example 5

### Selection of SV40 High Producing SuperVero Subclones

Puromycin-resistant Vero clones transfected with pAM001 and VERO-SF control cells were cultured until they reached a confluence of 50 percent. The cell cultures were transduced with 50 μl of the SV-Luc vector stock containing approximately 2.5×10¹⁰ vector genome copies.

Four hours post transduction the culture medium was replaced by fresh OptiPro SFM medium containing 2 μg/ml puromycine and 2 percent L-glutamine. Three days post transduction crude lysates are prepared from the transduced cells by freeze-thawing (Vera M. et al., Molecular Therapy 10: 780-791, 2004). COS-1 cells cultivated in DMEM supplemented with 10 percent fetal bovine serum (Invitrogen) were transduced with 100 microliters of crude lysate of each puromycin-resistant and pAM001-transfected Vero SF cell clone. Two days post transduction the COS-1 cells were subsequently tested for firefly luciferase expression as a measure for the amount of SV-Luc vector production in the corresponding puromycin-resistant and pAM001-transfected Vero SF cell clone. Cell clones that exhibited a comparable luciferase expression level to COS-1 cells were selected, propagated and expanded to create a cell bank. Cell clone Vero-SF001-86 was repeatedly monitored for SV-Luc production and produces similar amounts of recombinant SV40 vector particles as COS-1. A cell subclone of Vero-SF001-86 denoted Vero-SF001-86-01 was generated by limited dilution that repeatedly produces similar amounts of recombinant SV40 vector particles as the parental Vero-SF001-86 cell clone. Quantitative PCR according to Vera M. et al., Molecular Therapy 10: 780-791, 2004, revealed that cell clone Vero-SF001-86-01 denoted SuperVero routinely produces 1-10×10¹¹ vector genome copies per milliliter cell culture.

### Example 7

### Molecular Cloning of a Vector Used for Production of Recombinant Proteins in SuperVero Cells

The SV40 origin of replication was PCR isolated from pTracer-SV40 (Invitrogen) and cloned into the firefly luciferase expression plasmid pGL3 (Promega resulting in expression vector pAM006. Subsequently, SuperVero cells were transfected with purified pAM006 and the control pGL3 expression vector DNA. Three days after transfection luciferase expression was measured. SuperVero cells transfected with pAM6 produced significantly more firefly luciferase compared to the control pGL3 transfected cells.

### Example 8

### Construction of an Expression Plasmid Encoding the SV40 Large T Antigen

A synthetic multiple cloning site (MCS) was designed containing restriction sites for NotI, PacI, SbfI, PmeI, AscI and ClaI. Two oligonucleotides were designed WdV436: 5′-GCCGCTTTATTAATTAAGCCCTGCAGGTTGTTTAAACTTGGCGCGCCTTAT-3′ (SEQ ID NO: 18) and WdV437: 3′-CGATAAGGCGCGCCAAGTTTAAACAACCTGCAGGGCTTAATTAATAAAGC-5′. (SEQ ID NO 19). Both oligonucleotides WdV436 and WdV437 were annealed to each other and ligated into pBluescript SK-(Promega), yielding the recombinant plasmid pAM007.

Two oligonucleotides were designed to introduce an additional NotI restriction site WdV452: CGGCGGCCGCGTAC (SEQ ID NO: 20) and WdV453: GCGGCCGC. Both oligonucleotides were annealed and ligated into pAM007, yielding the recombinant vector pAM008.

The expression vector pLenti6.3/V5DEST_verA (Invitrogen) was used as a template for cloning of the cytomegalovirus immediate early (CMVie) promoter using PCR. Two oligonucleotides were designed WdV286: 5′-TTGGCGCGCCTCAATATTGGCCATTAGCCATATTATTCATTGG-3′ (SEQ ID NO: 21) and WdV220: 3′-GACAAGCTTCCAATGCACCGTTCCCGGCCGCGGAGGCTGGATCG-5′ (SEQ ID NO: 22) flanking the CMV promoter. Oligonucleotides WdV286 and WdV220 contained restriction sites Ascl and Hindlll respectively. Subsequently, purified pLenti6.3/V5DEST_verA was subjected to PCR using oligonuleotides WdV286 and WdV220, yielding a CMV promoter DNA fragment. This fragment was Ascl and Hindlll digested and ligated into pBluescript SK-, yielding pAM009.

The expression vector pGL4.22 (Promega) was used as a template for cloning of the puromycin N-acetyltransferase antibiotic resistance gene using PCR. Two oligonucleotides were designed WdV454: 5′-CCACCCAAGCTTATGACCGAGTACAAGCCCACGGTGCG-3′ (SEQ ID NO: 23) and WdV455: 3′-TATCCGCTCGAGTCAGGCACCGGGCTTGCGGGTCATGC-5′ (SEQ ID NO: 24) flanking the puromycin N-acetyltransferase antibiotic resistance gene and containing restriction sites HindIII and XhoI, respectively. Plasmid pGL4.22 was subjected to PCR using oligonucleotides WdV454 and WdV455, yielding the puromycin N-acetyltransferase cDNA. This fragment was HindIII and XhoI digested and ligated into pAM009, yielding pAM010.

The expression vector pEF5/FRT/5-DEST (Invitrogen) was used as a template for cloning of the BGH polyadenylation signal using PCR. Two oligonucleotides were designed WdV456: 5′-CAACCGCTCGAGCTGTGCCTTCTAGTTGCCAGCCATC-3′ (SEQ ID NO: 25) and WdV457: 3′-CGGGGTACCCCATAGAGCCCACCGCATCCCC-5′ (SEQ ID NO: 26) flanking the polyadenylation signal and containing restriction sites XhoI and KpnI respectively. Plasmid pEF5/FRT/V5-DEST was subjected to PCR using oligonucleotides WdV456 and WdV457, yielding the BGH polyadenylation signal cDNA. This fragment was Xhol and Kpnl digested and ligated into pAM010, yielding pAM011.

Plasmids pAM008 was digested with AscI and PmeI and the DNA fragment comprising the puromycin N-acetyltransferase coding domain was purified from an agarose gel and ligated into pAM008, yielding pAM012.

DNA of a full-length SV40 DNA clone (ATCC number VRMC-2) was used as template for cloning of the SV40 T antigen coding region using PCR. Two oligonucleotides were designed WdV408: ACCATGGATAAAGTTTTAAACAGAGAGGAATCTTTGCAGC (SEQ ID NO: 27) containing an attB1 recombination site and WdV409: TTATGTTTCAGGTTCAGGGGGAGGTGTGGGAGG (SEQ ID NO: 28) containing an attB2 recombination site. WdV408 and WdV409 were used to PCR amplify the genomic T antigen coding region. Subsequently, a gateway entry clone was generated from the generated DNA fragment and pDONR221, resulting in pAM013. A T antigen expression plasmid was generated by gateway recombination between pAM013 and pEF5/FRT/V5-DEST, resulting in pAM014.

The NotI and PmeI restriction sites in plasmid pAM014 were eliminated by NotI and PmeI digestion of pAM014 followed by a T4 DNA polymerase treatment and re-ligation, yielding pAM015. The T antigen expression cassette was subsequently isolated by a SphI digestion followed by a T4 DNA polymerase treatment and a Nrul digestion.

In order to generate a shuttle plasmid two oligonucleotides were designed

WdV448:
(SEQ ID NO: 29)
TCCTGCAGGCGGGGTACCCTAGTCTAGACTAGCCGCGGGGAGTTTAAACA
GCT
and
WdV449:
(SEQ ID NO: 30)
GTTTAAACTCCCCGCGGCTAGTCTAGACTAGGGTACCCCGCCTGCAGGAG
TAC.

Oligonucleotides WdV448 and WdV449 were annealed generating a DNA fragment that contains the KpnI, SbfI, KpnI, XbaI, SacII, PmeI and SacI restriction sites. This DNA fragment was ligated into KpnI and SacI digested pBluescript SK-(Promega), yielding pAM016. Plasmid pBluescript SK- was digested with KpnI and XbaI and the MCS DNA fragment was isolated from an agarose gel. The MCS DNA fragment was ligated into pAM016 digested with KpnI and XbaI, resulting in pAM017.

The EF1 alpha driven T antigen expression cassette from pAM015 was isolated by a NruI and SphI digest followed by a T4 DNA polymerase treatment. The resulting DNA fragment was cloned into pAM017 digested with EcoRV, resulting in pAM018.

Plasmid pAM018 was digested with Sbfl and Pmel and the DNA fragment comprising the T antigen expression cassette was isolated from an agarose gel and cloned into pAM012 digested with Sbfl and Pmel, resulting in pAM019.

Four oligonucleotides were designed

WdV487:
(SEQ ID NO: 31)
5′-GCAGGCTACCATGGATAAAGTTTTAAACAGAGAG-3′
and
WdV490:
(SEQ ID NO: 32)
3′-CCATTCATCAGTTCCATAGGTTGGAATCTCAGTTGCATCCCAGAAGC
CTCCAAAG-5′
WdV:489
(SEQ ID NO: 33)
5′CTTTGGAGGCTTCTGGGATGCAACTGAGATTCCAACCTATGGAACTGA
TGAATGGG-3′
and
WdV488:
(SEQ ID NO: 34)
5′-AGGAATGTTGTACACCATGCATTTTAAAAAGTC-3′.

Oligonuleotides WdV487 and WdV490 and oligonucleotides WdV489 and WdV488 were used to amplify the first and the second exon of the SV40 large T antigen respectively. Both generated DNA fragments were subsequently subjected to a fusion PCR using oligonucleotides WdV487 and WdV488.

The generated DNA fragment comprising the SV40 large T antigen coding region was digested with Ncol and Nsil and cloned into likewise digested pAM019, resulting in pAM001.

In summary, pAM001 contains an EF1 alpha promoter upstream of the large T antigen coding region and a CMVie promoter upstream of the puromycin N-acetyltransferase coding region.

### Example 9

### Amelioration of Clinical EAE Symptoms by Prophylactic and Therapeutic SVMOG Administration in Marmoset (Calliihrix jacchus)

A proof of concept study was conducted in which six bone marrow-chimeric twins were treated in a prophylactic setting: of each twin one sibling received one intravenous administration of the MOG encoding vector (SVMOG) and the other the control vector encoding luciferase (SV Luc). The animals were administered with 1×10¹¹ vector genomes. Subsequently, EAE was induced in marmosets using peptide, consisting of amino acid 34 to 56 of MOG (MOG34-56) in incomplete Freund's adjuvant (IFA; Difco Laboratories; Detroit Mich.) (A. Billiau and P. Matthys, Journal of Leukocyte Biology 70:849-860, 2001).

In parallel, EAE was induced in six bone marrow-chimeric twins using MOG34-56 in incomplete Freund's adjuvant. Subsequently, the animals were treated in a therapeutic setting: of each twin one sibling received one intravenous administration of SVMOG and the other sibling was administered with the control vector SVLuc. The animals were administered with 1×10¹¹ vector genomes.

The resulting disease course was characterized by quantifying lesions with inflammation and demyelination within the central nervous system (CNS) white and grey matter. This new and highly refined model shares essential clinical, radiological and pathological similarities with human MS, and has been described in detail in previous publications (Bert A. 't Hart et al., Trends in Molecular Medicine 17:119-25, 2011; Y. S. Kap et al., Journal of Neuroimmune Pharmacology 5:220-30, 2010). The severity of EAE was scored daily by two independent observers on a quantitative scale (Table 1).

TABLE 1
Integrated discomfort scoring table

| Discomfort Score | Clinical signs | Monitoring | Maximal duration | |
|---|---|---|---|---|
| 0 | Asymptomatic | Daily | End of experiment | |
| | No general discomfort signs | | | |
| 0.5 | Reduced alertness, loss of appetite, altered | Daily | 20 | weeks |
| | walking pattern without ataxia | | | |
| 1 | lethargy and/or weight loss less than 15% | Daily | 10 | weeks |
| | from start weight | | | |
| 2 | Ataxia (= reduced capacity to keep | Daily | 6 | weeks |
| | Balance), visual disturbance, optic neuritis | | | |
| 2.5 | Incomplete paralysis: | Daily | <7 | days |
| | para- or monoparesis and/or sensory loss | | | |
| | and/or brain stem syndrome | | | |
| 3.0 | Complete paralysis | Daily | <3 | days |
| | hind part of the body one- (hemiplegia) or | | | |
| | two-sided (paraplegia) | | | |
| 4.0^{#} | Complete paralysis | Daily | <18 | hours |
| | all four limbs quadriplegia | | | |
| 5.0^{#} | Lethargy (no reaction to external stimuli); | Daily | <1 | hour |
| | incapacity to eat or drink without help, self | | | |
| | mutilation, blindness more than two days, | | | |
| | untreatable pain | | | |

#: EAE scores 4 and 5 are normally not observed as they are beyond the ethical end-point. However, rarely the disease progresses very fast. Therefore, expression of scores 4 and 5 can be avoided.

It was found that prophylactic and therapeutic treatment of marmosets with SVMOG significantly ameliorates the development of EAE symptoms. It was also found that there was no adverse immune response against the antigen MOG during the time of observation when the antigen was expressed under transcriptional control of the SV40 early and late promoter, whereas a vast immune response was observed against MOG in animals immunized with MOG34-56 in incomplete Freund's adjuvant.

### Example 6

### Amelioration of Disease Symptoms of Other Neurological Degenerative Diseases by Prophylactic and Therapeutic Administration of a Replication-Deficient SV40 Vector Encoding a Major Auto-Antigen in Mouse (Mus musculus)

Recombinant SV40 vector DNA encoding one or more auto-antigens or parts thereof selected from the group consisting of neurofilament light (NFL) chain, alpha-actinin, glutamate receptor type 3 (GluR3), endothelial cell growth factor, acetylcholine receptor, hypocretin/orexin, aquaporin 4, and other auto-antigens identified using the confirmatory methods described herein above or using other immunological methods known to those skilled in the art, may be constructed after obtaining the DNA encoding the auto-antigens or parts thereof as described in Example 3 using chemical synthesis (GENEART®)). The DNA fragment may be subsequently recombined into pSVac-dest (pAM006) by GATEWAY® technology (Invitrogen) resulting in a pSVac expression plasmid DNA used to produce and purify SVac particles encoding the auto- as described in Example 4. These vector particles may be intravenously administered in animal model relevant to human neurological degenerative diseases such as Alzheimer's dementia, amyotrophic lateral sclerosis, bipolar disorder, depression, epilepsy, Huntington's disease, Lyme disease, multiple system atrophy, myasthenia gravis, narcolepsy, neuromyelitis optica, Parkinson's disease and cchizophrenia. In one embodiment the animals may be treated in a prophylactic setting, while in another embodiment the animals may be treated in a therapeutic setting as described in Example 5. The therapeutic effect of SVac administration is then evaluated for its ability to ameliorate the development of the neurological degenerative disease symptoms. Moreover, the adverse immune response against the auto-antigen may be monitored over time. It will be found that prophylactic and therapeutic treatment of animals with a SVac vector encoding a major auto-antigen involved in the autoimmune neural tissue destruction significantly ameliorates the development of disease symptoms. It will also be found that there is no adverse immune response against the auto-antigen during the time of observation when the auto-antigen is expressed under transcriptional control of the SV40 early and late promoter.

### Example 10

### Amelioration of Disease Symptoms of Metabolic Degenerative Diseases by Prophylactic and Therapeutic Administration of a Replication-Deficient SV40 Vector Encoding a Major Auto-Antigen in Mouse (Mus musculus)

Recombinant SV40 vector DNA encoding one or more auto-antigens or parts thereof selected from the group consisting of proinsulin, glutamic acid decarboxylase 65, thyroglobulin, golgi SNAP receptor complex member 1 (GOSR1), and other auto-antigens identified using the confirmatory methods described herein above or using other immunological methods known to those skilled in the art, may be constructed after obtaining the DNA encoding the auto-antigens or parts thereof as described in Example 3 using chemical synthesis (GENEART®). The DNA fragment may subsequently be recombined into pSVac-dest (pAM006) by GATEWAY® technology (Invitrogen) resulting in a pSVac expression plasmid DNA used to produce and purify SVac particles encoding the auto-antigens as described in Example 4. These vector particles may be intravenously administered in animal model relevant to human metabolic degenerative diseases such as type 1 diabetes, type 2 diabetes, Hashimoto's thyroiditis and obesity. In one embodiment the animals may be treated in a prophylactic setting, while in another embodiment the animals may be treated in a therapeutic setting as described in Example 5. The therapeutic effect of SVac administration may be evaluated for its ability to ameliorate the development of the metabolic degenerative disease symptoms. Moreover, the adverse immune response against the auto-antigen may be monitored over time. It will be found that prophylactic and therapeutic treatment of animals with a SVac vector encoding a major auto-antigen involved in the autoimmune tissue destruction significantly ameliorates the development of disease symptoms. It may also be found that there is no adverse immune response against the auto-antigen during the time of observation when the auto-antigen was expressed under transcriptional control of the SV40 early and late promoter.

### Example 11

### Amelioration of Disease Symptoms of Ophthalmological Degenerative Diseases by Prophylactic and Therapeutic Administration of a Replication-Deficient SV40 Vector Encoding a Major Auto-Antigen in Mouse (Mus musculus)

Recombinant SV40 vector DNA encoding one or more auto-antigens or parts thereof selected from the group consisting of arrestin, type I collagen, interphotoreceptor retinoid-binding protein, and other auto-antigens identified using the confirmatory methods described herein above or using other immunological methods known to those skilled in the art, may be constructed after obtaining the DNA encoding the auto-antigens or parts thereof as described in Example 3 using chemical synthesis (GENEART®). The DNA fragment may be subsequently recombined into pSVac-dest (pAM006) by GATEWAY® technology (Invitrogen) resulting in a pSVac expression plasmid DNA used to produce and purify SVac particles encoding the auto-antigens as described in Example 4. These vector particles may be intravenously administered in animal model relevant to human ophthalmological degenerative diseases such as autoimmune retinopathy, autoimmune macular degeneration, glaucoma, uveitis, retinitis pigmentosa, Leber's congenital amaurosis, Stargardt macular dystrophy, achromatopsia, retinoschisis and vitelliform macular dystrophy. In one embodiment the animals may be treated in a prophylactic setting, while in another embodiment the animals may treated in a therapeutic setting as described in Example 5. The therapeutic effect of SVac administration may be evaluated for its ability to ameliorate the development of the ophthalmological degenerative disease symptoms. Moreover, the adverse immune response against the auto-antigen may be monitored over time. It will be found that prophylactic and therapeutic treatment of animals with a SVac vector encoding a major auto-antigen involved in the autoimmune retinal tissue destruction significantly ameliorates the development of disease symptoms. It will also be found that there is no adverse immune response against the auto-antigen during the time of observation when the auto-antigen was expressed under transcriptional control of the SV40 early and late promoter.

### Example 12

### Amelioration of Disease Symptoms of Gastro-Enteric Degenerative Diseases by Prophylactic and Therapeutic Administration of a Replication-Deficient SV40 Vector Encoding a Major Auto-Antigen in Mouse (Mus musculus)

Recombinant SV40 vector DNA encoding one or more auto-antigens or parts thereof selected from the group consisting of transglutaminase, forminotransferase cyclodeaminase and other auto-antigens identified using the confirmatory methods described herein above or using other immunological methods known to those skilled in the art, may be constructed after obtaining the DNA encoding the auto-antigens or parts thereof as described in Example 3 using chemical synthesis (GENEART®). The DNA fragment may subsequently be recombined into pSVac-dest (pAM006) by GATEWAY® technology (Invitrogen) resulting in a pSVac expression plasmid DNA used to produce and purify SVac particles encoding the auto-antigens as described in Example 4. These vector particles may be intravenously administered in animal model relevant to human gastro-enteric degenerative diseases such as celiac disease, autoimmune hepatitis and inflammatory bowel diseases including Crohn' s disease and ulcerative colitis. In one embodiment the animals may be treated in a prophylactic setting, while in another embodiment the animals may be treated in a therapeutic setting as described in Example 5. The therapeutic effect of SVac administration may be evaluated for its ability to ameliorate the development of the gastro-enteric degenerative disease symptoms. Moreover, the adverse immune response against the auto-antigen may be monitored over time. It will be found that prophylactic and therapeutic treatment of animals with an SVac vector encoding a major auto-antigen involved in the autoimmune tissue destruction significantly ameliorates the development of disease symptoms. It will also be found that there is no adverse immune response against the auto-antigen during the time of observation when the auto-antigen was expressed under transcriptional control of the SV40 early and late promoter.

### Example 13

### Amelioration of Disease Symptoms of Dermatological Degenerative Diseases by Prophylactic and Therapeutic Administration of a Replication-Deficient SV40 Vector Encoding a Major Auto-Antigen in Mouse (Mus musculus)

Recombinant SV40 vector DNA encoding one or more auto-antigens or parts thereof selected from the group consisting of keratin, alpha-fodrin, and other auto-antigens identified using the confirmatory methods described herein above or using other immunological methods known to those skilled in the art, may be constructed after obtaining the DNA encoding the auto-antigens or parts thereof as described in Example 3 using chemical synthesis (GENEART®). The DNA fragment may subsequently be recombined into pSVac-dest (pAM006) by GATEWAY® technology (Invitrogen) resulting in a pSVac expression plasmid DNA used to produce and purify SVac particles encoding the auto-antigens as described in Example 4. These vector particles may be intravenously administered in animal model relevant to human dermatological degenerative diseases such as psoriasis, scleroderma, Sjögren's syndrome and vitiligo. In one embodiment the animals may be treated in a prophylactic setting, while in another embodiment the animals may be treated in a therapeutic setting as described in Example 5. The therapeutic effect of SVac administration may be evaluated for its ability to ameliorate the development of the dermatological degenerative disease symptoms. Moreover, the adverse immune response against the auto-antigen may be monitored over time. It will be found that prophylactic and therapeutic treatment of animals with an SVac vector encoding a major auto-antigen involved in the autoimmune skin destruction significantly ameliorates the development of disease symptoms. It will also be found that there is no adverse immune response against the auto-antigen during the time of observation when the auto-antigen was expressed under transcriptional control of the SV40 early and late promoter.

### Example 14

### Amelioration of Disease Symptoms of Cardio-vascular Degenerative Diseases by Prophylactic and Therapeutic Administration of a Replication-Deficient SV40 Vector Encoding a Major Auto-Antigen in Mouse (Mus musculus)

Recombinant SV40 vector DNA encoding one or more auto-antigens or parts thereof selected from the group consisting of elastin, beta-2-glycoprotein I, and other auto-antigens identified using the confirmatory methods described herein above or using other immunological methods known to those skilled in the art, may be constructed after obtaining the DNA encoding the auto-antigens or parts thereof as described in Example 3 using chemical synthesis (GENEART®). The DNA fragment may subsequently be recombined into pSVac-dest (pAM006) by GATEWAY® technology (Invitrogen) resulting in a pSVac expression plasmid DNA used to produce and purify SVac particles encoding the auto-antigens as described in Example 4. These vector particles may be intravenously administered in animal model relevant to human cardio-vascular degenerative diseases such as atherosclerosis, cardiomyopathy and Coxsackie myocarditis. In one embodiment the animals may be treated in a prophylactic setting, while in another embodiment the animals may be treated in a therapeutic setting as described in Example 5. The therapeutic effect of SVac administration may be evaluated for its ability to ameliorate the development of the cardio-vascular degenerative disease symptoms. Moreover, the adverse immune response against the auto-antigen may be monitored over time. It will be found that prophylactic and therapeutic treatment of animals with an SVac vector encoding a major auto-antigen involved in the autoimmune endothelial tissue destruction significantly ameliorates the development of disease symptoms. It will also be found that there is no adverse immune response against the auto-antigen during the time of observation when the auto-antigen was expressed under transcriptional control of the SV40 early and late promoter.

### Example 15

### Amelioration of Disease Symptoms of Orthopedic Degenerative Diseases by Prophylactic and Therapeutic Administration of a Replication-Deficient SV40 Vector Encoding a Major Auto-Antigen in Mouse (Mus musculus)

Recombinant SV40 vector DNA encoding one or more auto-antigens or parts thereof selected from the group consisting of RNA-induced silencing complex (RISC) components, type II collagen, and other auto-antigens identified using the confirmatory methods described herein above or using other immunological methods known to those skilled in the art, may be constructed after obtaining the DNA encoding the auto-antigens or parts thereof as described in Example 3 using chemical synthesis (GENEART®). The DNA fragment may subsequently be recombined into pSVac-dest (pAM006) by GATEWAY® technology (Invitrogen) resulting in a pSVac expression plasmid DNA used to produce and purify SVac particles encoding the auto-antigens as described in Example 4. These vector particles may be intravenously administered in animal model relevant to human orthopedic degenerative diseases such as rheumatoid arthritis, rheumatic fever and lupus erythematosus. In one embodiment the animals may be treated in a prophylactic setting, while in another embodiment the animals may be treated in a therapeutic setting as described in Example 5. The therapeutic effect of SVac administration may be evaluated for its ability to ameliorate the development of the orthopedic degenerative disease symptoms. Moreover, the adverse immune response against the auto-antigen may be monitored over time. It will be found that prophylactic and therapeutic treatment of animals with an SVac vector encoding a major auto-antigen involved in the autoimmune tissue destruction significantly ameliorates the development of disease symptoms. It will also be found that there is no adverse immune response against the auto-antigen during the time of observation when the auto-antigen was expressed under transcriptional control of the SV40 early and late promoter.

### Example 16

### Amelioration of Disease Symptoms of Muscular Degenerative Diseases by Prophylactic and Therapeutic Administration of a Replication-Deficient SV40 Vector Encoding a Major Auto-Antigen in Mouse (Mus musculus)

Recombinant SV40 vector DNA encoding one or more auto-antigens or parts thereof such as histidyl-tRNA-synthetase, and other auto-antigens identified using the confirmatory methods described herein above or using other immunological methods known to those skilled in the art, may be constructed after obtaining the DNA encoding the auto-antigens or parts thereof as described in Example 3 using chemical synthesis (GENEART®). The DNA fragment may subsequently be recombined into pSVac-dest (pAM006) by GATEWAY® technology (Invitrogen) resulting in a pSVac expression plasmid DNA used to produce and purify SVac particles encoding the auto-antigens as described in Example 4. These vector particles may be intravenously administered in animal model relevant to human muscular degenerative diseases such as polymyositis, dermatomyositis, inclusion body myositis, Duchenne muscular dystrophy, Becker muscular dystrophy, Miyoshi myopathy, limb-girdle muscular dystrophy, congenital muscular dystrophy, distal muscular dystrophy, Emery-Dreyfuss muscular dystrophy, fascio-scapulohumeral muscular dystrophy, myotonic muscular dystrophy and oculopharyngeal muscular dystrophy. In one embodiment the animals may be treated in a prophylactic setting, while in another embodiment the animals may be treated in a therapeutic setting as described in Example 5. The therapeutic effect of SVac administration may be evaluated for its ability to ameliorate the development of the muscular degenerative disease symptoms. Moreover, the adverse immune response against the auto-antigen may be monitored over time. It will be found that prophylactic and therapeutic treatment of animals with an SVac vector encoding a major auto-antigen involved in the autoimmune muscle destruction significantly ameliorates the development of disease symptoms. It will also be found that there is no adverse immune response against the auto-antigen during the time of observation when the auto-antigen was expressed under transcriptional control of the SV40 early and late promoter.

### Example 17

### Amelioration of Disease Symptoms of Pulmonary Degenerative Diseases by Prophylactic and Therapeutic Administration of a Replication-Deficient SV40 Vector Encoding a Major Auto-Antigen in Mouse (Mus musculus)

Recombinant SV40 vector DNA encoding one or more auto-antigens or parts thereof selected from the group consisting of elastin, beta-2-glycoprotein I, and other auto-antigens identified using the confirmatory methods described herein above or using other immunological methods known to those skilled in the art, may be constructed after obtaining the DNA encoding the auto-antigens or parts thereof as described in Example 3 using chemical synthesis (GENEART®). The DNA fragment may subsequently be recombined into pSVac-dest (pAM006) by GATEWAY® technology (Invitrogen) resulting in a pSVac expression plasmid DNA used to produce and purify SVac particles encoding the auto-antigens as described in Example 4. These vector particles may be intravenously administered in animal model relevant to human pulmonary degenerative diseases such as chronic obstructive pulmonary disease and asthma. In one embodiment the animals may be treated in a prophylactic setting, while in another embodiment the animals may be treated in a therapeutic setting as described in Example 5. The therapeutic effect of SVac administration may be evaluated for its ability to ameliorate the development of the pulmonary degenerative disease symptoms. Moreover, the adverse immune response against the auto-antigen may be monitored over time. It will be found that prophylactic and therapeutic treatment of animals with an SVac vector encoding a major auto-antigen involved in the autoimmune lung destruction significantly ameliorates the development of disease symptoms. It will also be found that there is no adverse immune response against the auto-antigen during the time of observation when the auto-antigen was expressed under transcriptional control of the SV40 early and late promoter.

### Example 18

### Testing the Ability of Viral Promoters to Induce RNA Interference

The VP35 open reading frame from Ebola virus strain Zaire was cloned into the mammalian expression vector pEF5-V5-DEST containing the human EF1-alpha promoter using GATEWAY® technology (Invitrogen) as previously described (J. Haasnoot et al., Plos Pathogens 3:e86, 2007). Human embryonic kidney (HEK293T) cells were grown as a monolayer in DMEM (Gibco) supplemented with 10 percent of fetal bovine serum (FBS) (Gibco) at 37 degrees Celsius and 5 percent of CO₂. One day before transfection, cells were trypsinized, resuspended in DMEM, and seeded in 24-well plates at a density of 1.5×10⁵ cells per well. At the time of transfection, the cells were 60 to 70 percent confluent. The transfection was performed in quadruplicates using LIPOFECTAMINE® 2000 (Invitrogen) according to the instructions of the manufacturer. For the luciferase and renilla assay, cells were co-transfected with increasing amounts (0, 10, 50, 100 nanograms) of expression construct encoding the RNA silencing suppressor VP35 and either 2 nanograms of expression plasmid encoding the Renilla luciferase under the control of CMV immediate early promoter (pRL-CMV), 100 nanograms of expression plasmid encoding the firefly luciferase under the control of SV40 early promoter (pGL3) or 100 nanograms of expression plasmid encoding the firefly luciferase under transcriptional control of the full-length SV40 early and late promoter (SVLuc, SEQ ID NO: 12). Lysates from cultured cells were prepared 2 days after transfection as follows: Cell culture medium was removed and cells were rinsed with 100 microliters of phosphate buffered saline (PBS). Cells were then lysed in 100 microliters of lx Passive Lysis Buffer (PLB, Promega) by shaking for 30 minutes at room temperature. Firefly or Renilla luciferase expression was measured in 10 microliters of supernatant with the dual luciferase reporter assay system (Promega).

HEK293T cells transfected with plasmids where reporter gene expression was driven by the CMV immediate early promoter or the SV40 early promoter (pRL-CMV and pGL3), in the presence of an expression plasmid encoding a viral suppressor of RNA interference; VP35, accumulate high amounts of reporter protein in a dose-dependent manner (FIGS. 1A and 1B, respectively). HEK293T cells transfected with plasmids where reporter gene expression was driven by the full-length SV40 early and late promoter (SVLuc, SEQ ID NO: 12) accumulated high amounts of reporter gene independently of the presence or absence of VP35 (FIG. 1C). From these experiments it may be concluded that the CMV immediate early promoter and the SV40 early promoter induce RNA interference. The full-length SV40 early and late promoter does not induce RNA interference in mammalian cells.

The invention claimed is:

1. Method for the production of recombinant polyomaviral vector particles not encoding a functional polyomaviral small T antigen, the method comprising:
providing a non-human primate SV40 permissive cell or cell line, wherein the SV40 permissive cell or cell line comprises a gene encoding a functional polyomaviral large T antigen stably integrated into the genome of the cell; and
wherein the SV40 permissive cell or cell line does not comprise a gene encoding a functional polyomaviral small T antigen,
introducing into the SV40 permissive cell or cell line a polyomavirus DNA not encoding a functional polyomaviral small T antigen,
culturing the cell or cell line in a growth medium under conditions allowing the formation of recombinant polyomaviral vector particles, and
harvesting the recombinant polyomaviral vector particles from the cell culture.

2. A composition comprising recombinant polyomaviral vector particles not encoding a functional polyomaviral small T antigen produced by the method according to claim 1.

3. The composition of claim 2, wherein the composition comprises more than one million recombinant polyomaviral vector particles not encoding a functional polyomaviral small T antigen, the polyomaviral vector particles being incapable of replicating in cells that are permissive for the wildtype polyomavirus and do not express a functional polyomaviral large T antigen, wherein the composition does not contain a single polyomavirus particle being able to replicate in cells which are permissive for the wildtype polyomavirus wherein the cells do not express a functional polyomaviral large T antigen.

4. The composition of claim 3 wherein the polyomavirus is a primate polyomavirus.

5. The composition of claim 4 wherein the polyomavirus is a simian polyomavirus.

6. The composition of claim 5 wherein the polyomavirus is selected from the group consisting of SV40, SV12, Lymphotropic polyomavirus, African green monkey polyomavirus and Chimpanzee polyomavirus.

7. The composition of claim 6 wherein the polyomavirus is SV40.

8. The composition of claim 3, wherein the SV40 permissive cell or cell line is selected from the group consisting of Vero cells and CV1.

9. A non-human primate SV40 permissive cell or cell line, the cell or cell line comprising the recombinant polyomaviral vector particles of claim 2;
wherein the cell or cell line comprises a gene encoding a functional polyomaviral large T antigen stably integrated into the genome of the cell; and
wherein the cell or cell line does not comprise a gene a functional polyomaviral small T antigen.

10. A method for the production of a recombinant protein, the method comprising:
utilizing the cell line of claim 9 to produce a recombinant protein.

11. The composition of claim 9, wherein the SV40 permissive cell or cell line is selected from the group consisting of Vero cells and CV1.

12. A method of inducing immune tolerance toward an auto-antigen in a host organism without inducing RNA interference to mRNA encoding the auto-antigen, the method comprising:
administering to the host organism a replication-defective SV40 vector comprising a polynucleotide encoding the auto-antigen under the transcriptional control of a full-length SV40 early and late promoter comprising SEQ ID NO: 1 or SEQ ID NO: 12; and
determining that there is no adverse immune response to expression of the auto-antigen;
wherein expression of mRNA from the polynucleotide in the cells of the host organism does not induce RNA interference against the expressed mRNA; and
wherein expression of the auto-antigen induces immune tolerance toward the auto-antigen in the host organism.
